(19) United States
(12) Patent Application Publication
(10) Pub. No.: US 2025/0115622 A1
(43) Pub. Date: Apr. 10, 2025

(54) Crystalline Forms Of An MCL-1 Inhibitor

(71) Applicant: AMGEN INC., Thousand Oaks, CA (US)

(72) Inventors: Ron C. Kelly, Thousand Oaks, CA (US); Mary Chaves, Arlington, MA (US); Jing Teng, Thousand Oaks, CA (US); Stephan Parent, Thousand Oaks, CA (US); Markian Stec, Thousand Oaks, CA (US); Van Luu, Thousand Oaks, CA (US); Robert P. Farrell, Thousand Oaks, CA (US); James E. Huckle, Thousand Oaks, CA (US); Michal Achmatowicz, Simi Valley, CA (US); Tian Wu, Newbury Park, CA (US); Darren Leonard Reid, Belmont, MA (US); Lingyun Xiao, Thousand Oaks, CA (US)

(21) Appl. No.: 18/730,357

(22) PCT Filed: Feb. 3, 2023
(86) PCT No.: PCT/US23/12251

§ 371 Date: Jul. 19, 2024

Related U.S. Application Data
(60) Provisional application No. 63/306,776, filed on Feb. 4, 2022.

(51) Int. Cl. C07D 519/00; A61K 31/553

(52) U.S. Cl. CPC C07D 519/00; A61K 31/553

(57) ABSTRACT

Disclosed herein are crystalline forms of (4S,7aR,9aR,10R,11E,14S,15R)-6′-chloro-10-methoxy-14,15-dimethyl-10-{[(9aR)-octahydro-2H-pyrido[1,2-a]pyrazin-2-yl]methyl}-3′,4′,7a,8,9,9a,10,13,14,15-decahydro-2′H,3H,5H-spiro[1,19-etheno-1616-cyclobuta[i] [1,4]oxazepino[3,4-f][1,2,7]thiadiazacyclohexadecine-4,1′-naphthalene]-16,16,18(7H,17H)-trione (AMG 397): (AMG 397), hydrates, and solvates thereof. Also disclosed are methods of making the crystalline forms, and methods of treating diseases and disorders with the crystalline forms.

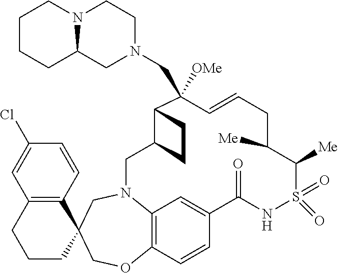

### BACKGROUND

### Technical Field

The present disclosure relates to crystalline forms of (4S,7aR,9aR,10R,11E,14S,15R)-6′-chloro-10-methoxy-14,15-dimethyl-10-{[(9aR)-octahydro-2H-pyrido[1,2-a]pyrazin-2-yl]methyl}-3′,4′,7a,8,9,9a,10,13,14,15-decahydro-2′H,3H,5H-spiro[1,19-etheno-1616-cyclobuta[i][1,4]oxazepino[3,4-f][1,2,7]thiadiazacyclohexadecine-4,1′-naphthalene]-16,16,18(7H,17H)-trione (AMG 397), hydrates, and solvates thereof, which functions as an inhibitor of myeloid cell leukemia 1 protein (Mcl-1).

### Description of Related Technology

The compound, (4S,7aR,9aR,10R,11E,14S,15R)-6′-chloro-10-methoxy-14,15-dimethyl-10-{[(9aR)-octahydro-2H-pyrido[1,2-a]pyrazin-2-yl]methyl}-3′,4′,7a,8,9,9a,10,13,14,15-decahydro-2′H,3H,5H-spiro[1,19-etheno-1616-cyclobuta[i][1,4]oxazepino[3,4-f][1,2,7]thiadiazacyclohexadecine-4,1′-naphthalene]-16,16,18(7H,17H)-trione (AMG 397), is useful as an inhibitor of myeloid cell leukemia 1 (“Mcl-1):

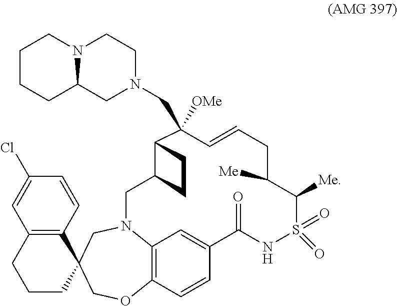

One common characteristic of human cancer is overexpression of Mcl-1. Mcl-1 overexpression prevents cancer cells from undergoing programmed cell death (apoptosis), allowing the cells to survive despite widespread genetic damage.

Mcl-1 is a member of the Bcl-2 family of proteins. The Bcl-2 family includes pro-apoptotic members (such as BAX and BAK) which, upon activation, form a homo-oligomer in the outer mitochondrial membrane that leads to pore formation and the escape of mitochondrial contents, a step in triggering apoptosis. Antiapoptotic members of the Bcl-2 family (such as Bcl-2, Bcl-XL, and Mcl-1) block the activity of BAX and BAK. Other proteins (such as BID, BIM, BIK, and BAD) exhibit additional regulatory functions. Research has shown that Mcl-1 inhibitors can be useful for the treatment of cancers. MCI-1 is overexpressed in numerous cancers.

U.S. Pat. No. 10,300,075, which is incorporated herein by reference in its entirety, discloses AMG 397 as an Mcl-1 inhibitor and provides a method for preparing it. However, alternative forms of AMG 397 with improved properties are desirable, particularly for clinical use of AMG 397.

### SUMMARY

Provided herein are crystalline forms of AMG 397, hydrates, and solvates thereof, wherein AMG 397 has the structure

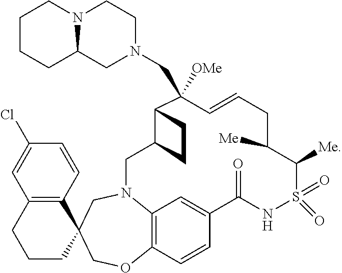

Also provided herein are crystalline forms of AMG 397 as a hydrate, characterized by solid state ¹³C NMR peaks at 5.65, 15.29, 18.06, 21.54, 24.20, 24.87, 28.91, 29.87, 36.86, 37.74, 39.09, 43.79, 44.59, 48.25, 49.01, 51.76, 54.33, 55.45, 57.50, 60.39, 64.99, 66.40, 80.11, 82.55, 83.01, 115.39, 121.81, 124.57, 127.61, 129.92, 132.04, 133.60, 135.32, 140.41, 142.61, 143.54, 153.09, 173.18, and 174.17±0.5 ppm (“Form 2 hydrate”).

Also provided herein are crystalline forms of AMG 397 as a hydrate, characterized by solid state ¹³C NMR peaks at 7.07, 17.2, 21.14, 22.75, 23.74, 27.01, 27.79, 29.13, 30.12, 32.09, 33.0, 35.45, 37.96, 45.21, 45.88, 50.0, 54.43, 55.23, 57.5, 59.23, 61.66, 63.31, 64.14, 69.06, 76.48, 82.72, 116.84, 119.24, 121.1, 126.62, 130.68, 132.8, 136.76, 139.39, 140.98, 141.7, 151.61, 172.8, and 173.61±0.5 ppm (“Form 3 hydrate”).

Also provided herein are crystalline forms of AMG 397 anhydrous, characterized by solid state ¹³C NMR peaks at 5.55, 17.86, 24.02, 24.95, 29.56, 37.70, 44.44, 47.61, 48.86, 51.26, 54.92, 56.72, 57.48, 58.58, 64.86, 82.34, 114.99, 121.30, 127.31, 131.61, 133.04, 135.02, 139.77, 141.92, 152.71, and 173.08±0.5 ppm (“Form 4 anhydrous”).

Also provided herein are crystalline forms of AMG 397 as a hydrate, characterized by solid state ¹³C NMR peaks at 5.90, 15.93, 21.71, 24.33, 24.99, 25.92, 28.37, 29.16, 30.25, 31.00, 37.10, 39.31, 44.09, 48.49, 49.30, 51.99, 54.58, 55.81, 56.34, 57.73, 60.59, 66.60, 80.42, 83.22, 115.55, 122.14, 124.75, 127.82, 130.10, 132.40, 133.76, 140.62, 142.89, 143.63, 153.36, and 174.41±0.5 ppm (“Form 5 hydrate”).

Also provided herein are crystalline forms of AMG 397 anhydrous, characterized by XRPD pattern peaks at 8.3, 15.7, 16.0, 18.6, and 20.1±0.2° 2θ using Cu Kα radiation (“Form 6 anhydrous”).

Also provided herein are crystalline forms of AMG 397 as a hydrate, characterized by XRPD pattern peaks at 8.3, 10.7, and 10.8±0.2° 2θ using Cu Kα radiation (“Form 7 hydrate”).

Also provided herein are crystalline forms of AMG 397 as an ethanol solvate, characterized by XRPD pattern peaks at 9.9, 16.9, and 20.0±0.2° 2θ using Cu Kα radiation (“Form 8 ethanol solvate”).

Also provided herein are crystalline forms of AMG 397 as a hydrate, characterized by XRPD pattern peaks at 10.0, 17.0, and 20.2±0.2° 2θ using Cu Kα radiation (“Form 9 hydrate”).

Also provided herein are crystalline forms of AMG 397 as a hydrate, characterized by XRPD pattern peaks at 10.1, 20.2, 20.3±0.2° 2θ using Cu Kα radiation (“Form 10 hydrate”).

Also provided herein are pharmaceutical formulations comprising the crystalline forms of AMG 397, and hydrates and solvates thereof, as described herein and a pharmaceutically acceptable excipient.

Also provided herein are methods of treating a subject suffering from cancer, comprising administering to the subject a therapeutically effective amount of the pharmaceutical formulation comprising the crystalline forms of AMG 397, and hydrates and solvates thereof as described herein and a pharmaceutically acceptable excipient.

### BRIEF DESCRIPTION OF THE DRAWINGS

FIG. 1 depicts an X-ray powder diffraction (“XRPD”) pattern of amorphous AMG 397.

FIG. 2 depicts a differential scanning calorimetry (“DSC”) thermograph of amorphous AMG 397 indicating a Tg of 195.90° C.

FIG. 3 depicts a thermogravimetric analysis (“TGA”) trace of amorphous AMG 397 showing 0.86% weight loss to 175° C. prior to degradation.

FIG. 4 depicts a moisture sorption profile (DVS) of amorphous AMG 397 showing weight gain of ˜6.4% by 95% relative humidity.

FIG. 5 depicts an X-ray powder diffraction (“XRPD”) pattern of the crystalline hydrate form 1 of AMG 397.

FIG. 6 depicts a differential scanning calorimetry (“DSC”) thermograph of the crystalline hydrate form 1 of AMG 397 indicating a Tm of 221° C.

FIG. 7 depicts a thermogravimetric analysis (“TGA”) trace of the crystalline hydrate form 1 of AMG 397 showing 2.0% weight loss to ˜200° C., prior to melt/degradation. Single crystal structure confirmed variable hydrate, water amount from 0.6%-2% observed.

FIG. 8 depicts a moisture sorption profile (DVS) of the crystalline hydrate form 1 of AMG 397 showing weight gain of ˜3.3% by 95% relative humidity.

FIG. 9 depicts a solid state ¹³C NMR of the crystalline hydrate form 1 of AMG 397.

FIG. 10 depicts a single crystal X-ray crystal structure of crystalline hydrate form 1 of AMG 397.

FIG. 11 depicts an X-ray powder diffraction (“XRPD”) pattern of the crystalline hydrate form 2 of AMG 397.

FIG. 12 depicts a differential scanning calorimetry (“DSC”) thermograph of the crystalline hydrate form 2 of AMG 397 indicating a Tm of 248° C.

FIG. 13 depicts a thermogravimetric analysis (“TGA”) trace of the crystalline hydrate form 2 of AMG 397 showing 1.8% weight loss to 225° C., prior to melt/degradation.

FIG. 14 depicts a moisture sorption profile (DVS) of the crystalline hydrate form 2 of AMG 397 showing weight gain of ˜3.0% by 95% relative humidity.

FIG. 15 depicts a solid state ¹³C NMR of the crystalline hydrate form 2 of AMG 397.

FIG. 16 depicts an X-ray powder diffraction (“XRPD”) pattern of the crystalline hydrate form 3 of AMG 397.

FIG. 17 depicts a differential scanning calorimetry (“DSC”) thermograph of the crystalline hydrate form 3 of AMG 397 indicating a Tm of 237° C.

FIG. 18 depicts a thermogravimetric analysis (“TGA”) trace of the crystalline hydrate form 3 of AMG 397 showing 6.2% weight loss to 230° C., prior to melt/degradation.

FIG. 19 depicts a moisture sorption profile (DVS) of the crystalline hydrate form 3 of AMG 397 showing weight gain of ˜1.9% by 95% relative humidity.

FIG. 20 depicts a solid state ¹³C NMR of the crystalline hydrate form 3 of AMG 397.

FIG. 21 depicts an X-ray powder diffraction (“XRPD”) pattern of the crystalline anhydrous form 4 of AMG 397.

FIG. 22 depicts a differential scanning calorimetry (“DSC”) thermograph of the crystalline anhydrous form 4 of AMG 397 indicating a Tm of 242° C.

FIG. 23 depicts a thermogravimetric analysis (“TGA”) trace of the crystalline anhydrous form 4 of AMG 397 showing 0.6% weight loss to 225° C., prior to melt/degradation.

FIG. 24 depicts a moisture sorption profile (DVS) of the crystalline anhydrous form 4 of AMG 397 showing weight gain of ˜4.5% by 95% relative humidity.

FIG. 25 depicts a solid state ¹³C NMR of the crystalline anhydrous form 4 of AMG 397.

FIG. 26 depicts an X-ray powder diffraction (“XRPD”) pattern of the crystalline hydrate form 5 of AMG 397.

FIG. 27 depicts a differential scanning calorimetry (“DSC”) thermograph of the crystalline hydrate form 5 of AMG 397 indicating a Tm 237° C.

FIG. 28 depicts a thermogravimetric analysis (“TGA”) trace of the crystalline hydrate form 5 of AMG 397 showing 2.3% weight loss to 225° C., prior to melt/degradation.

FIG. 29 depicts a solid state ¹³C NMR of the crystalline hydrate form 5 of AMG 397.

FIG. 30 depicts an X-ray powder diffraction (“XRPD”) pattern of the crystalline anhydrous form 6 of AMG 397.

FIG. 31 depicts a differential scanning calorimetry (“DSC”) thermograph of the crystalline anhydrous form 6 of AMG 397 indicating a Tm of 234° C.

FIG. 32 depicts a thermogravimetric analysis (“TGA”) trace of the crystalline anhydrous form 6 of AMG 397 showing 0.3% weight loss from 25-120° C., prior to melt/degradation.

FIG. 33 depicts a moisture sorption profile (DVS) of the crystalline anhydrous form 6 of AMG 397 showing weight gain of ˜0.5% between 0-50% relative humidity, and 10% 50-95% relative humidity.

FIG. 34 depicts an X-ray powder diffraction (“XRPD”) pattern of the crystalline hydrate form 7 of AMG 397.

FIG. 35 depicts a differential scanning calorimetry (“DSC”) thermograph of the crystalline hydrate form 7 of AMG 397. Hot stage microscopy confirms melt at 216.9-223.8° C.

FIG. 36 depicts a thermogravimetric analysis (“TGA”) trace of the crystalline hydrate form 7 of AMG 397 showing 4.15% weight loss to 150° C., prior to melt/degradation.

FIG. 37 depicts a moisture sorption profile (DVS) of the crystalline hydrate form 7 of AMG 397 showing variable water content from 0-12% wt.

FIG. 38 depicts an X-ray powder diffraction (“XRPD”) pattern of the crystalline ethanol solvate form 8 of AMG 397.

FIG. 39 depicts a differential scanning calorimetry (“DSC”) thermograph of the crystalline ethanol solvate form 8 of AMG 397 showing a Tm onset of 67° C. (peak 91° C.), and Tm onset of 236° C.

FIG. 40 depicts a thermogravimetric analysis (“TGA”) trace of the crystalline ethanol solvate form 8 of AMG 397 showing 31.3% weight loss between 37-140° C., prior to melt/degradation.

FIG. 41 depicts a single crystal X-ray crystal structure of crystalline ethanol solvate form 8 of AMG 397.

FIG. 42 depicts an X-ray powder diffraction (“XRPD”) pattern of the crystalline hydrate form 9 of AMG 397.

FIG. 43 depicts a differential scanning calorimetry (“DSC”) thermograph of the crystalline hydrate form 9 of AMG 397 showing Tm onset of 234° C.

FIG. 44 depicts a thermogravimetric analysis (“TGA”) trace of the crystalline hydrate form 9 of AMG 397 showing 1.8% weight loss between 37-130° C., prior to melt/degradation.

FIG. 45 depicts an X-ray powder diffraction (“XRPD”) pattern of the crystalline hydrate form 10 of AMG 397.

FIG. 46 depicts a differential scanning calorimetry (“DSC”) thermograph of the crystalline hydrate form 10 of AMG 397 showing Tm onset of 233° C.

FIG. 47 depicts a thermogravimetric analysis (“TGA”) trace of the crystalline hydrate form 10 of AMG 397 showing 1.63% weight loss between 25-220° C., prior to melt/degradation.

FIG. 48 depicts an overlay of XRPD Patterns of AMG 397 anhydrous and hydrate forms: (forms 1-9 from top to bottom).

FIG. 49 depicts unique XRPD peaks for AMG 397 anhydrous and hydrate forms.

FIG. 50 depicts an overlay of solid state ¹³C NMR traces of the crystalline anhydrous and hydrate forms 1-5 of AMG 397.

FIG. 51 depicts processes for form conversion of AMG 397 free base.

### DETAILED DESCRIPTION

Disclosed herein are crystalline forms of (4S,7aR,9aR,10R,11E,14S,15R)-6′-chloro-10-methoxy-14,15-dimethyl-10-{[(9aR)-octahydro-2H-pyrido[1,2-a]pyrazin-2-yl]methyl}-3′,4′,7a,8,9,9a,10,13,14,15-decahydro-2′H,3H,5H-spiro[1,19-etheno-1616-cyclobuta[i][1,4]oxazepino[3,4-f][1,2,7]thiadiazacyclohexadecine-4,1′-naphthalene]-16,16,18(7H,17H)-trione (AMG 397), and hydrates thereof:

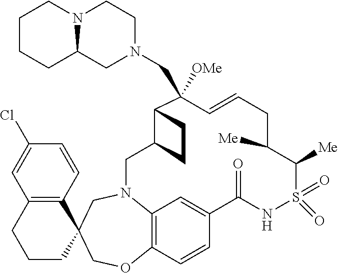

(AMG 397). AMG 397 anhydrous form 4 is a thermodynamically stable form. Hydrate forms of crystalline AMG 397, such as AMG 397 crystalline hydrate form 1, can be advantageous over AMG 397 anhydrous form 4 due to higher solubility, bioavailability and a robust crystallization process.

Also provided herein are pharmaceutical formulations of crystalline forms of AMG 397, and methods of treating a subject suffering from cancer, comprising administering to the subject a therapeutically effective amount of a pharmaceutical formulation of a crystalline form as disclosed herein.

U.S. Pat. No. 10,300,075, which is incorporated by reference herein in its entirety, discloses synthetic procedures for synthesizing Mcl-1 inhibitors, such as AMG 397.

Further provided herein are crystalline hydrate forms of AMG 397, pharmaceutical formulations thereof, and methods of treating a subject suffering from cancer, comprising administering to the subject a therapeutically effective amount of a pharmaceutical formulation of a crystalline hydrate form as disclosed herein.

The compounds disclosed herein may be identified either by their chemical structure and/or chemical name herein. When the chemical structure and chemical name conflict, the chemical structure is determinative of the identity of the compound.

When ranges are used herein for physical properties, such as molecular weight, or chemical properties, such as chemical formulae, all combinations and subcombinations of ranges and specific embodiments therein are intended to be included.

As used herein, chemical structures which contain one or more stereocenters depicted with dashed and bold bonds (i.e., [symbol] and [symbol] ) are meant to indicate absolute stereochemistry of the stereocenter(s) present in the chemical structure. As used herein, bonds symbolized by a simple line do not indicate a stereo-preference.

Unless otherwise indicated to the contrary, chemical structures that include one or more stereocenters which are illustrated herein without indicating absolute or relative stereochemistry encompass all possible stereoisomeric forms of the compound (e.g., diastereomers, enantiomers) and mixtures thereof. Structures with a single bold or dashed line, and at least one additional simple line, encompass a single enantiomeric series of all possible diastereomers.

The term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

“Treatment” or “treating” means any treatment of a disease in a patient, including: a) preventing the disease, that is, causing the clinical symptoms of the disease not to develop; b) inhibiting the disease; c) slowing or arresting the development of clinical symptoms; and/or d) relieving the disease, that is, causing the regression of clinical symptoms. Treatment of diseases and disorders herein is intended to also include the prophylactic administration of a pharmaceutical formulation described herein to a subject (i.e., an animal, preferably a mammal, most preferably a human) believed to be in need of treatment, such as, for example, cancer.

The term “therapeutically effective amount” means an amount effective, when administered to a human or non-human patient, to treat a disease, e.g., a therapeutically effective amount may be an amount sufficient to treat a disease or disorder responsive to inhibition of Mcl-1. The therapeutically effective amount may be ascertained experimentally, for example by assaying blood concentration of the chemical entity, or theoretically, by calculating bioavailability.

The term “solvate” refers to the chemical entity formed by the interaction of a solvate and a compound. Crystalline solvates of AMG 397 used in formulations herein are specifically contemplated. Solvents that can form crystalline solvate forms of AMG 397 include without limitation, ethanol. In some cases, a solvate has 0.5 to 2 solvent molecules per AMG 397 molecule.

The term “hydrate” is a specific type of solvate, where the solvent is water. A hydrate, as used herein, can have a variable amount of water including, e.g., hemi-hydrates, monohydrates, dihydrates, trihydrates, etc. Crystalline hydrates of AMG 397 are specifically contemplated for use in the formulations disclosed herein. In some cases, the hydrate has 0.5 to 2 water molecules pe AMG 397 molecule.

The term “polymorph” as used herein includes all crystalline and amorphous forms of the compound, including, for example, polymorphs, pseudopolymorphs, solvates, hydrates, unsolvated polymorphs (including anhydrates), and conformational polymorphs, as well as mixtures thereof, unless a particular crystalline or amorphous form is referred to. In some embodiments, the disclosure provides crystalline forms of AMG 397, for example, crystalline polymorphs, pseudopolymorphs, solvates, hydrates, unsolvated polymorphs (including anhydrates), and conformational polymorphs, as well as mixtures thereof, unless a particular crystalline form is referred to.

### Crystalline Forms

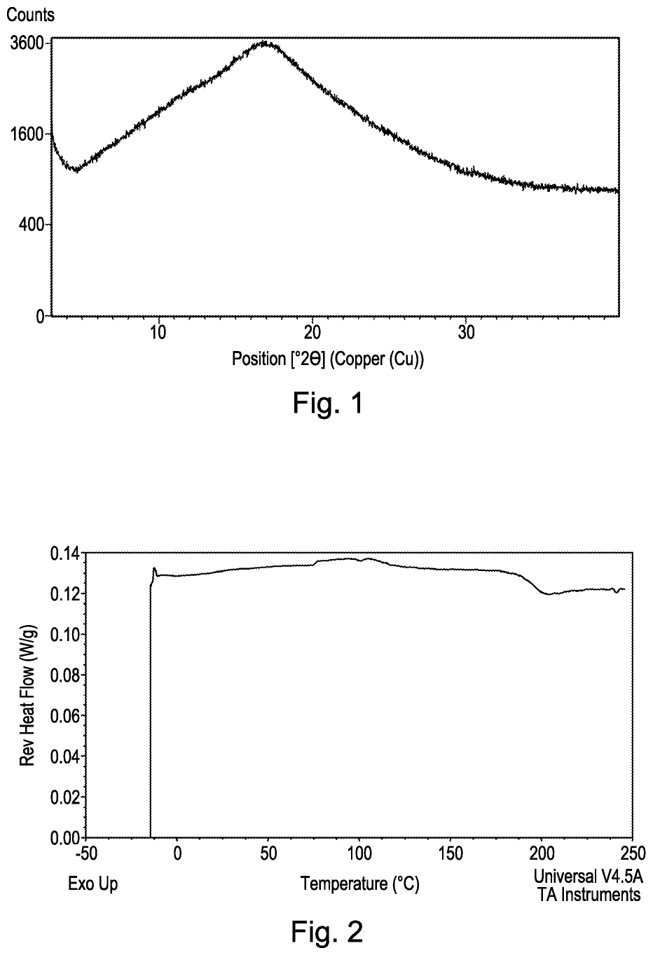

Amorphous Form: The amorphous form of AMG 397 can be characterized by X-ray powder diffraction, obtained as set forth in the Examples using Cu Kα radiation. In some embodiments, the amorphous form has an X-ray powder diffraction pattern substantially as shown in FIG. 1, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Differential scanning calorimetry (DSC) thermographs were obtained, as set forth in the Examples, for the amorphous form of AMG 397. The DSC curve indicates an endothermic transition at 195.9° C.±3° C. Thus, in some embodiments, the amorphous form of AMG 397 can be characterized by a DSC thermograph having a transition endotherm with an onset of 192.9° C. to 198.9° C. For example, in some embodiments the amorphous form of AMG 397 is characterized by DSC, as shown in FIG. 2.

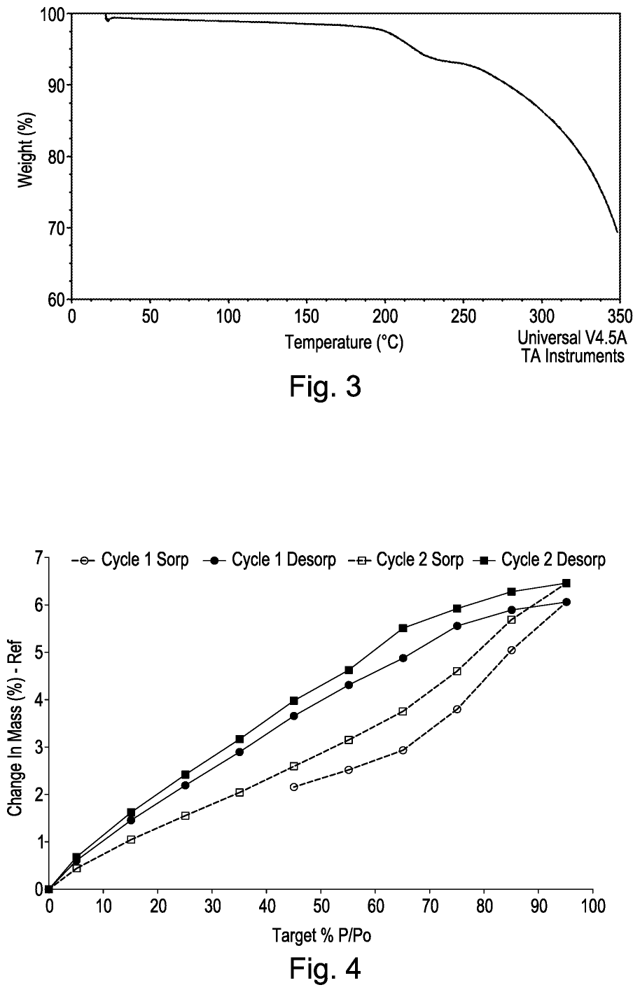

The amorphous form of AMG 397 can be characterized by thermogravimetric analysis (TGA). Thus, the amorphous form of AMG 397 can be characterized by a weight loss in a range of about 0% to about 0.86% with an onset temperature of about 175° C. In some embodiments, the amorphous form of AMG 397 has a thermogravimetric analysis substantially as depicted in FIG. 3, wherein by “substantially” is meant that the reported TGA features can vary by ±5° C.

The amorphous form of AMG 397 can be characterized by a moisture sorption profile. For example, in some embodiments, the amorphous form of AMG 397 is characterized by the moisture sorption profile as shown in FIG. 4, showing a weight gain of 6.4% by 95% RH.

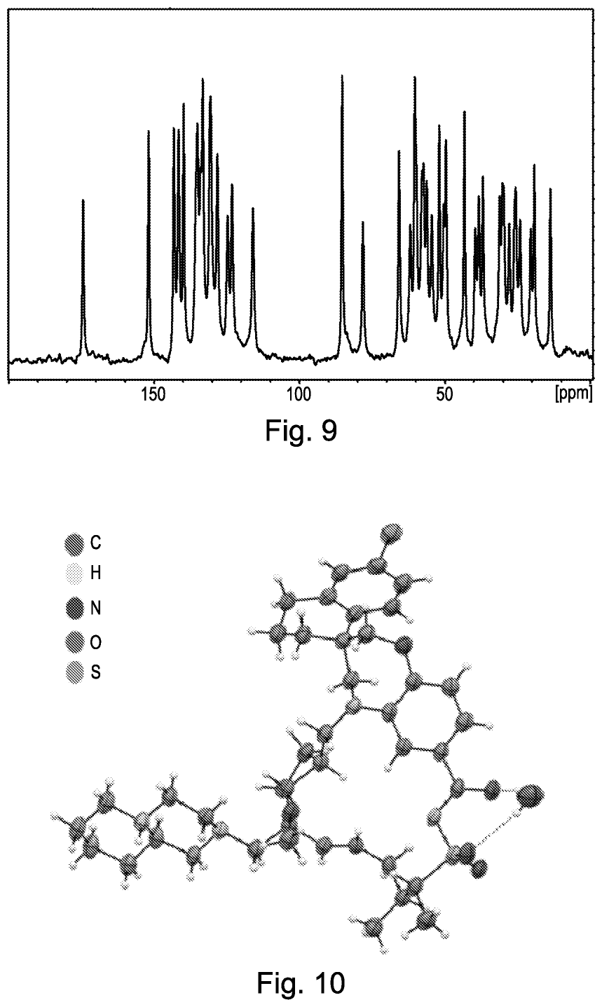

Hydrate Form 1: Hydrate form 1 can be characterized by solid state ¹³C NMR, obtained as set forth in the Examples, having peaks at 13.57, 19.13, 20.39, 24.04, 25.54, 27.75, 30.09, 31.05, 36.84, 38.27, 39.48, 43.15, 49.53, 50.30, 51.84, 54.40, 56.15, 57.28, 57.78, 60.23, 61.80, 65.65, 78.05, 85.23, 115.91, 123.10, 124.60, 128.11, 130.53, 133.18, 133.87, 134.99, 139.72, 141.47, 143.08, 151.76, and 174.30±0.5 ppm. In some embodiments, hydrate form 1 has a solid state ¹³C NMR substantially as shown in FIG. 9, wherein by “substantially” is meant that the reported peaks can vary by ±0.5 ppm.

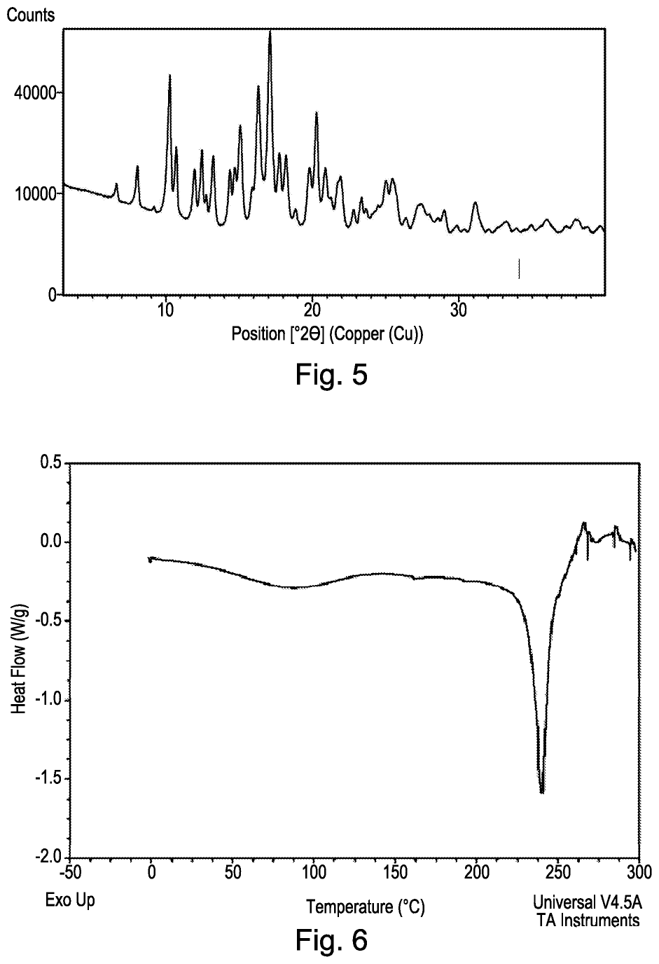

Hydrate form 1 can be characterized by an X-ray powder diffraction pattern, obtained as set forth in the Examples, having peaks at 10.3, 16.3, and 17.1±0.2° 2θ using Cu Kα radiation, optionally further characterized by additional peaks at 10.7, 12.5, 13.3, 15.1, 17.7, 18.2, and 20.3±0.2° 2θ using Cu Kα radiation, and/or additional peaks at 8.1, 12.0, 14.4, 14.7, 19.8, 20.9, 21.9, 25.0, and 25.4±0.2° 2θ using Cu Kα radiation. In some embodiments, hydrate form 1 has an X-ray powder diffraction pattern substantially as shown in FIG. 5, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Differential scanning calorimetry (DSC) thermographs were obtained, as set forth in the Examples, for hydrate form 1. The DSC curve indicates an endothermic transition at 221° C.±3° C. Thus, in some embodiments, hydrate form 1 can be characterized by a DSC thermograph having a transition endotherm with an onset of 218° C. to 224° C. For example, in some embodiments hydrate form 1 is characterized by DSC, as shown in FIG. 6.

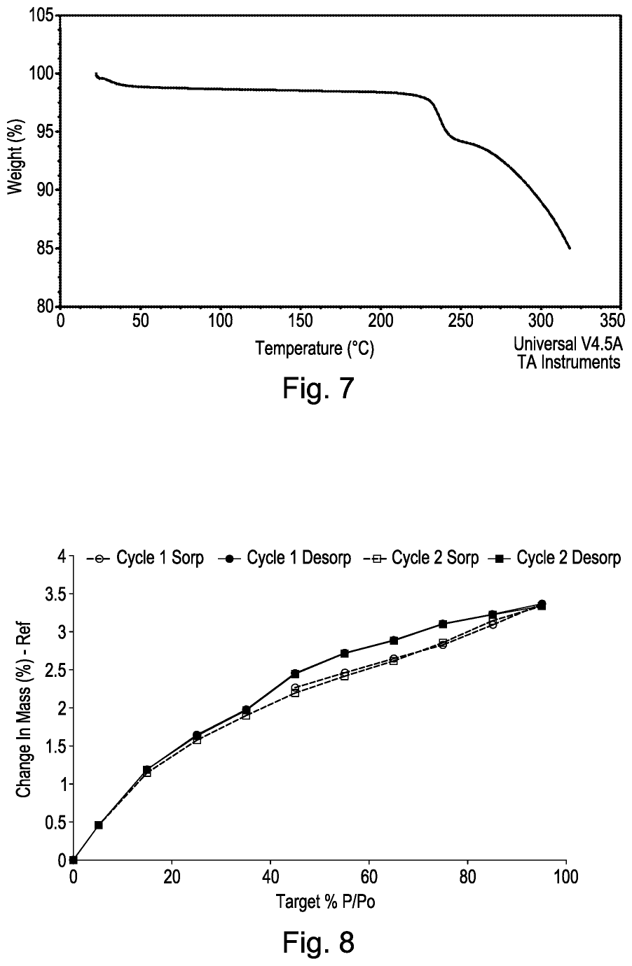

Hydrate form 1 can be characterized by thermogravimetric analysis (TGA). Thus, hydrate form 1 can be characterized by a weight loss in a range of about 0% to about 3% with an onset temperature of 218° C. to 224° C. For example, hydrate form 1 can be characterized by a weight loss of about 2%, up to about 200° C. In some embodiments, hydrate form 1 has a thermogravimetric analysis substantially as depicted in FIG. 7, wherein by “substantially” is meant that the reported TGA features can vary by ±5° C.

Hydrate form 1 can be characterized by a moisture sorption profile. For example, in some embodiments, hydrate form 1 is characterized by the moisture sorption profile as shown in FIG. 8, showing a weight gain of 3.3% by 95% RH.

Hydrate form 1 can be characterized by a single crystal structure substantially as shown in FIG. 10, or as set forth in the Examples.

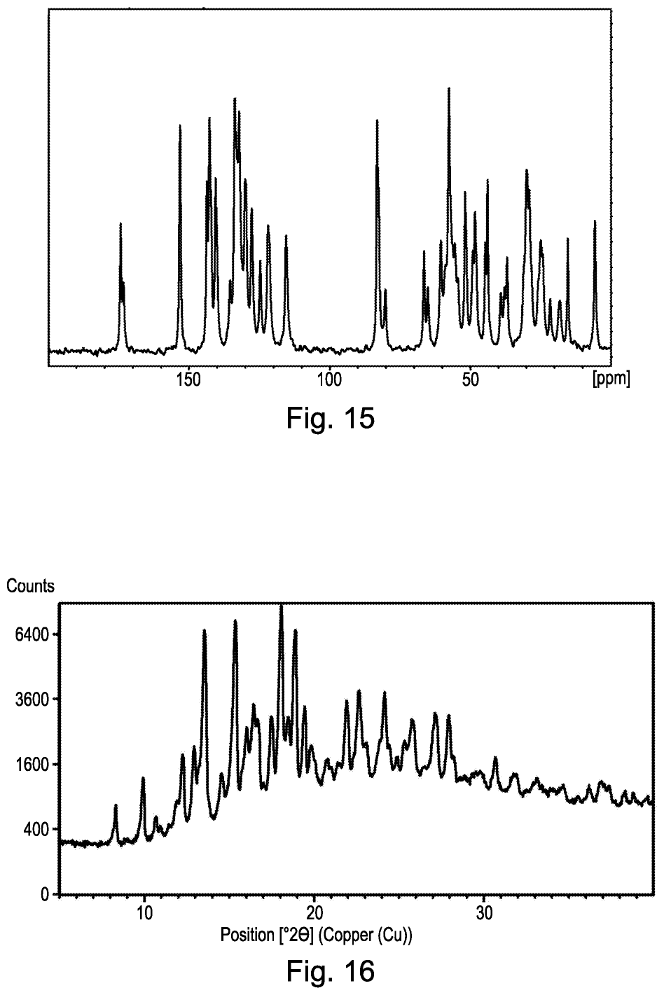

Hydrate Form 2: Hydrate form 2 can be characterized by solid state ¹³C NMR, obtained as set forth in the Examples, having peaks at 5.65, 15.29, 18.06, 21.54, 24.20, 24.87, 28.91, 29.87, 36.86, 37.74, 39.09, 43.79, 44.59, 48.25, 49.01, 51.76, 54.33, 55.45, 57.50, 60.39, 64.99, 66.40, 80.11, 82.55, 83.01, 115.39, 121.81, 124.57, 127.61, 129.92, 132.04, 133.60, 135.32, 140.41, 142.61, 143.54, 153.09, 173.18, and 174.17±0.5 ppm. In some embodiments, hydrate form 2 has a solid state ¹³C NMR substantially as shown in FIG. 15, wherein by “substantially” is meant that the reported peaks can vary by ±0.5 ppm.

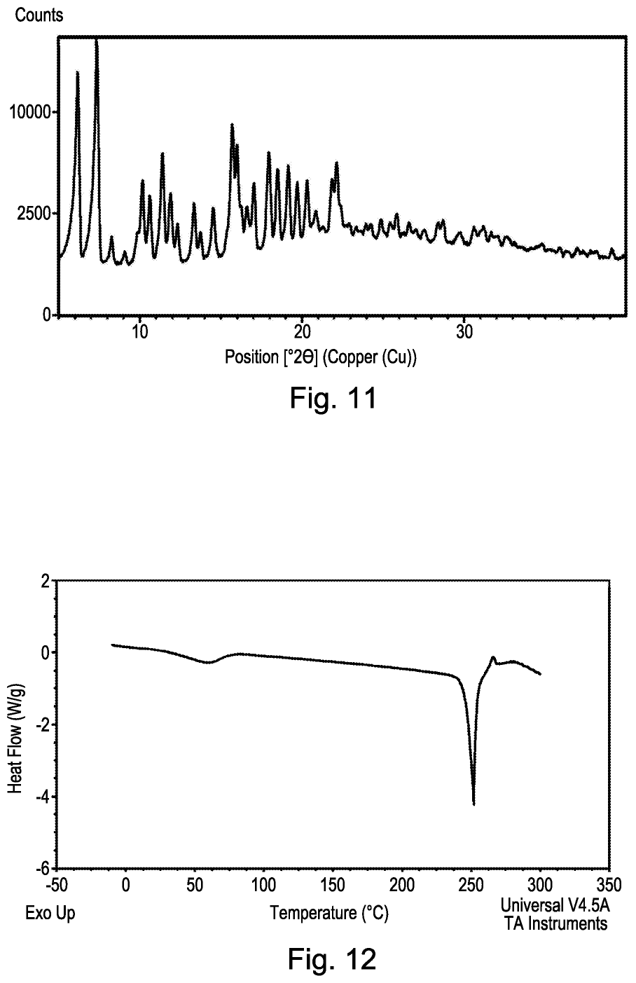

Hydrate form 2 can be characterized by an X-ray powder diffraction pattern, obtained as set forth in the Examples, having peaks at 6.2, 7.4, and 15.7±0.2° 2θ using Cu Kα radiation, optionally further characterized by additional peaks at 11.4, 16.0, 18.0, and 22.1±0.2° 2θ using Cu Kα radiation, and/or additional peaks at 10.2, 10.6, 11.9, 17.1, 18.5, 19.2, 19.7, 20.3, 20.9, and 21.8.0±0.2° 2θ using Cu Kα radiation. In some embodiments, hydrate form 2 has an X-ray powder diffraction pattern substantially as shown in FIG. 11, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

Differential scanning calorimetry (DSC) thermographs were obtained, as set forth in the Examples, for hydrate form 2. The DSC curve indicates an endothermic transition at 248° C.±3° C. Thus, in some embodiments, hydrate form 2 can be characterized by a DSC thermograph having a transition endotherm with an onset of 245° C. to 251° C. For example, in some embodiments hydrate form 2 is characterized by DSC, as shown in FIG. 12.

Hydrate form 2 can be characterized by thermogravimetric analysis (TGA). Thus, hydrate form 2 can be characterized by a weight loss in a range of about 0% to about 1.8% with an onset temperature of about 225° C.

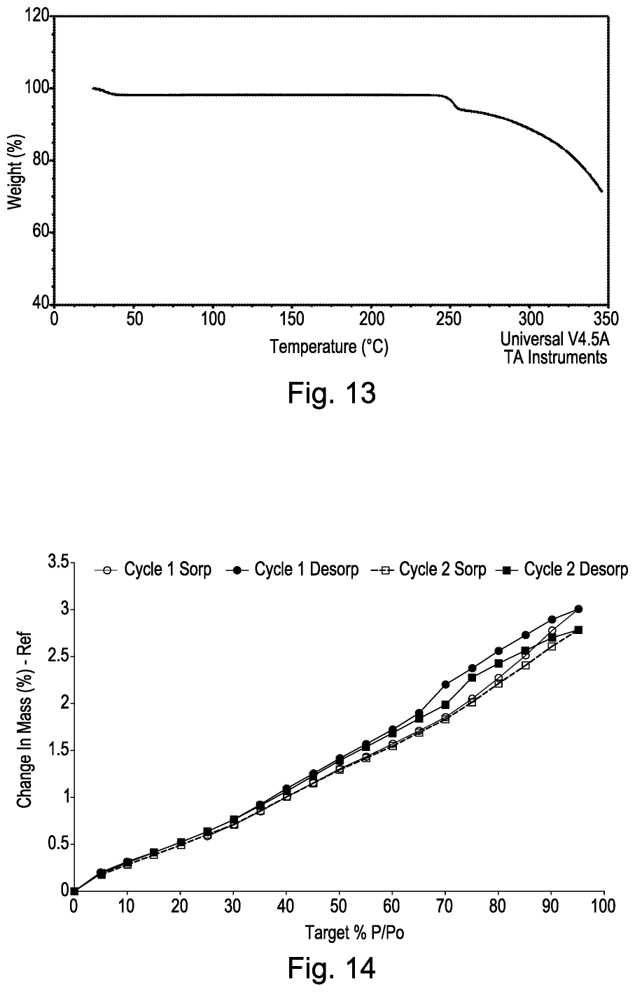

In some embodiments, hydrate form 2 has a thermogravimetric analysis substantially as depicted in FIG. 13, wherein by “substantially” is meant that the reported TGA features can vary by ±5° C.

Hydrate form 2 can be characterized by a moisture sorption profile. For example, in some embodiments, hydrate form 2 is characterized by the moisture sorption profile as shown in FIG. 14, showing a weight gain of 3% by 95% RH.

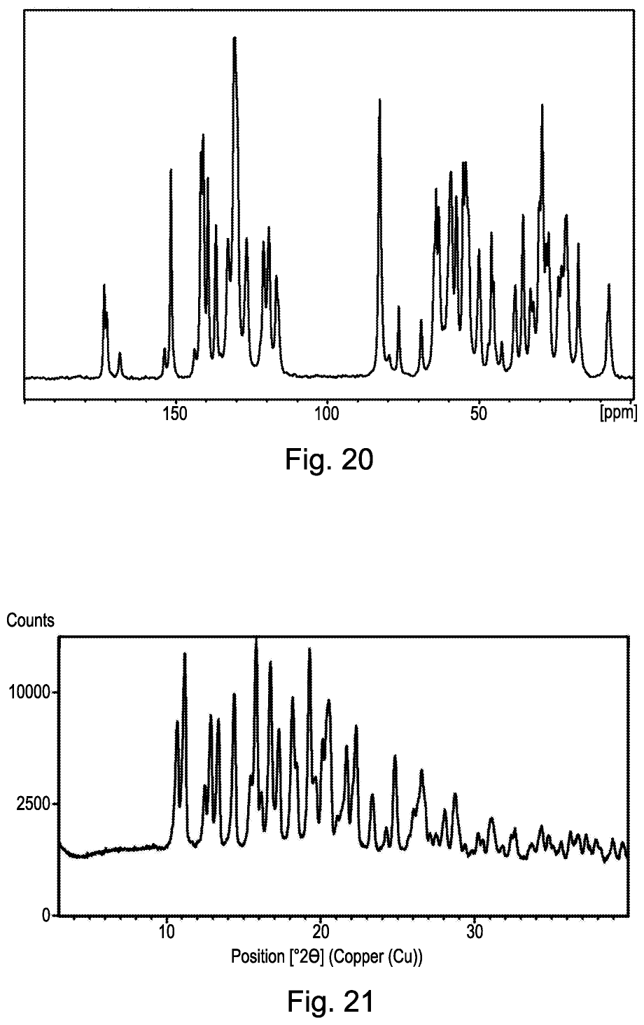

Hydrate Form 3: Hydrate form 3 can be characterized by solid state ¹³C NMR, obtained as set forth in the Examples, having peaks at 7.07, 17.2, 21.14, 22.75, 23.74, 27.01, 27.79, 29.13, 30.12, 32.09, 33.0, 35.45, 37.96, 45.21, 45.88, 50.0, 54.43, 55.23, 57.5, 59.23, 61.66, 63.31, 64.14, 69.06, 76.48, 82.72, 116.84, 119.24, 121.1, 126.62, 130.68, 132.8, 136.76, 139.39, 140.98, 141.7, 151.61, 172.8, and 173.61±0.5 ppm. In some embodiments, hydrate form 3 has a solid state ¹³C NMR substantially as shown in FIG. 20, wherein by “substantially” is meant that the reported peaks can vary by ±0.5 ppm.

Hydrate form 3 can be characterized by an X-ray powder diffraction pattern, obtained as set forth in the Examples, having peaks at 13.6, 15.4, and 18.1±0.2° 2θ using Cu Kα radiation, optionally further characterized by additional peaks at 16.5, 18.9, 21.9, 22.6, and 24.2±0.2° 2θ using Cu Kα radiation, and/or additional peaks at 12.3, 13.0, 16.0, 16.8, 17.5, 18.5, 19.5, 23.0, 27.2, and 28.0±0.2° 2θ using Cu Kα radiation. In some embodiments, hydrate form 3 has an X-ray powder diffraction pattern substantially as shown in FIG. 16, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

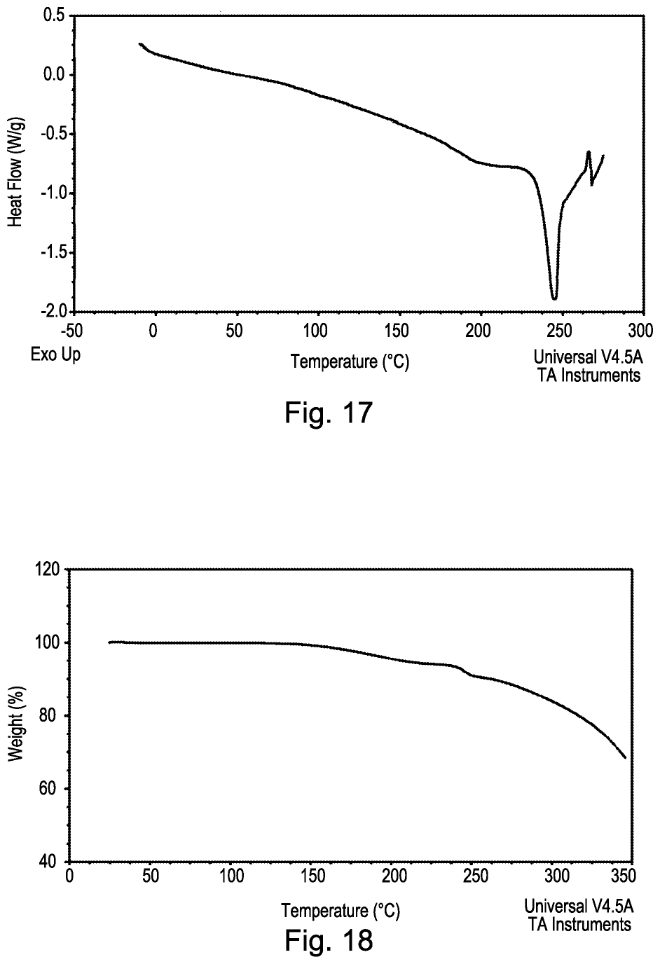

Differential scanning calorimetry (DSC) thermographs were obtained, as set forth in the Examples, for hydrate form 3. The DSC curve indicates an endothermic transition at 237° C.±3° C. Thus, in some embodiments, hydrate form 3 can be characterized by a DSC thermograph having a transition endotherm with an onset of 234° C. to 240° C. For example, in some embodiments hydrate form 3 is characterized by DSC, as shown in FIG. 17.

Hydrate form 3 can be characterized by thermogravimetric analysis (TGA). Thus, hydrate form 3 can be characterized by a weight loss in a range of about 0% to about 6.2% with an onset temperature of about 230° C.

In some embodiments, hydrate form 3 has a thermogravimetric analysis substantially as depicted in FIG. 18, wherein by “substantially” is meant that the reported TGA features can vary by ±5° C.

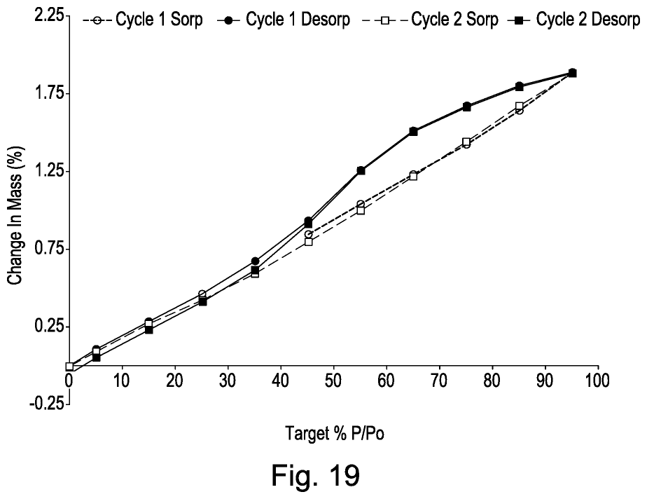

Hydrate form 3 can be characterized by a moisture sorption profile. For example, in some embodiments, hydrate form 3 is characterized by the moisture sorption profile as shown in FIG. 19, showing a weight gain of 1.9% by 95% RH.

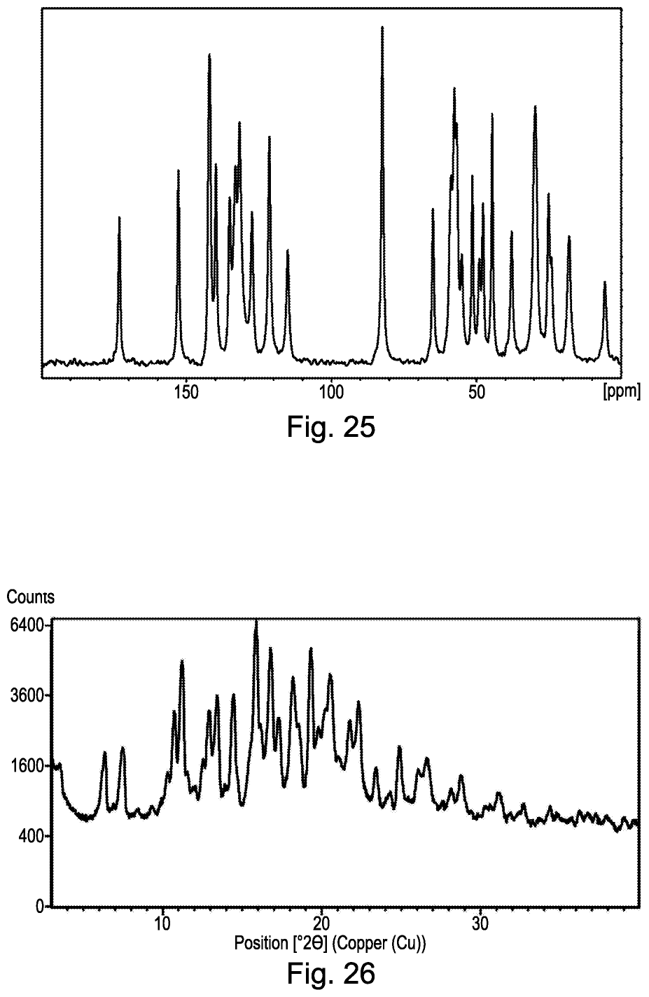

Anhydrous Form 4: Anhydrous form 4 can be characterized by solid state ¹³C NMR, obtained as set forth in the Examples, having peaks at 5.55, 17.86, 24.02, 24.95, 29.56, 37.70, 44.44, 47.61, 48.86, 51.26, 54.92, 56.72, 57.48, 58.58, 64.86, 82.34, 114.99, 121.30, 127.31, 131.61, 133.04, 135.02, 139.77, 141.92, 152.71, and 173.08±0.5 ppm. In some embodiments, anhydrous form 4 has a solid state ¹³C NMR substantially as shown in FIG. 25, wherein by “substantially” is meant that the reported peaks can vary by ±0.5 ppm.

Anhydrous form 4 can be characterized by an X-ray powder diffraction pattern, obtained as set forth in the Examples, having peaks at 11.2, 15.8, and 19.3±0.2° 2θ using Cu Kα radiation, optionally further characterized by additional peaks at 12.9, 14.4, 16.8, and 18.2±0.2° 2θ using Cu Kα radiation, and/or additional peaks at 10.7, 13.4, 15.4, 17.3, 18.5, 20.1, 20.4, 20.6, 21.7, 22.3, 24.9, and 26.5±0.2° 2θ using Cu Kα radiation. In some embodiments, anhydrous form 4 has an X-ray powder diffraction pattern substantially as shown in FIG. 21, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

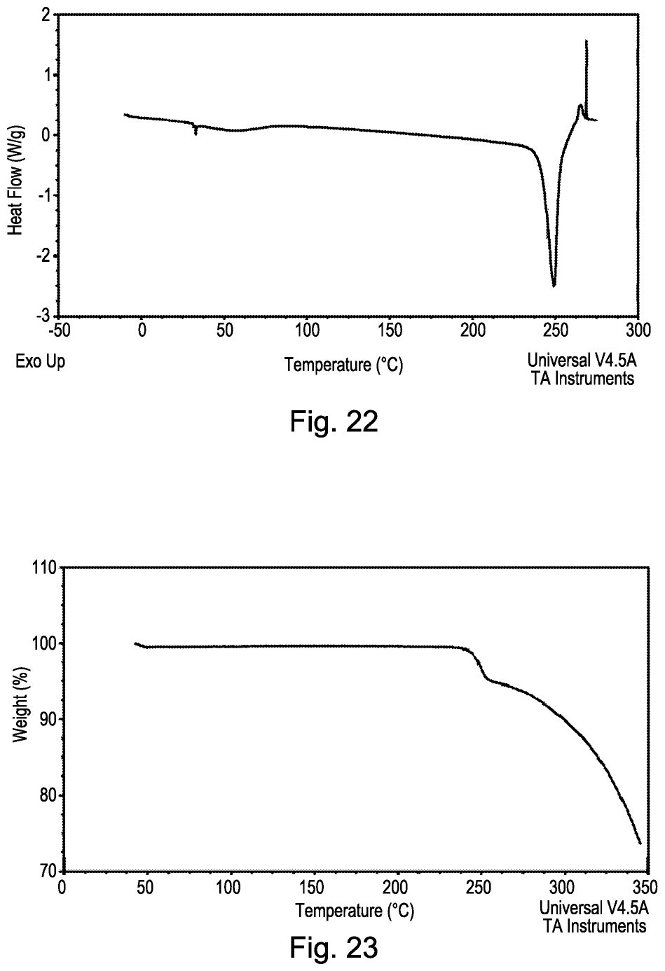

Differential scanning calorimetry (DSC) thermographs were obtained, as set forth in the Examples, for anhydrous form 4. The DSC curve indicates an endothermic transition at 242° C.±3° C. Thus, in some embodiments, anhydrous form 4 can be characterized by a DSC thermograph having a transition endotherm with an onset of 239° C. to 245° C. For example, in some embodiments anhydrous form 4 is characterized by DSC, as shown in FIG. 22.

Anhydrous form 4 can be characterized by thermogravimetric analysis (TGA). Thus, anhydrous form 4 can be characterized by a weight loss in a range of about 0% to about 0.6% with an onset temperature of about 225° C. In some embodiments, anhydrous form 4 has a thermogravimetric analysis substantially as depicted in FIG. 23, wherein by “substantially” is meant that the reported TGA features can vary by ±5° C.

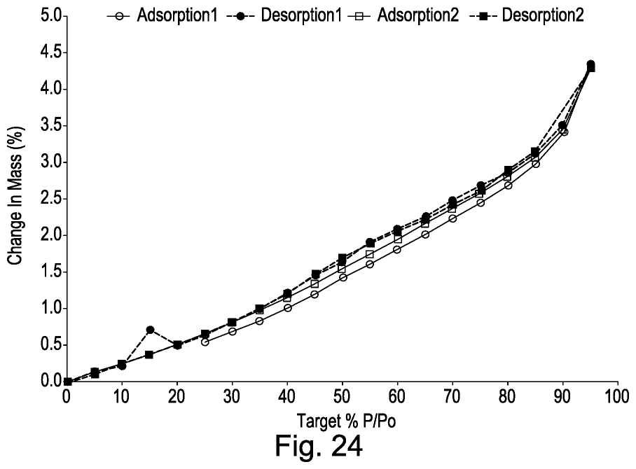

Anhydrous form 4 can be characterized by a moisture sorption profile. For example, in some embodiments, anhydrous form 4 is characterized by the moisture sorption profile as shown in FIG. 24, showing a weight gain of 4.5% by 95% RH.

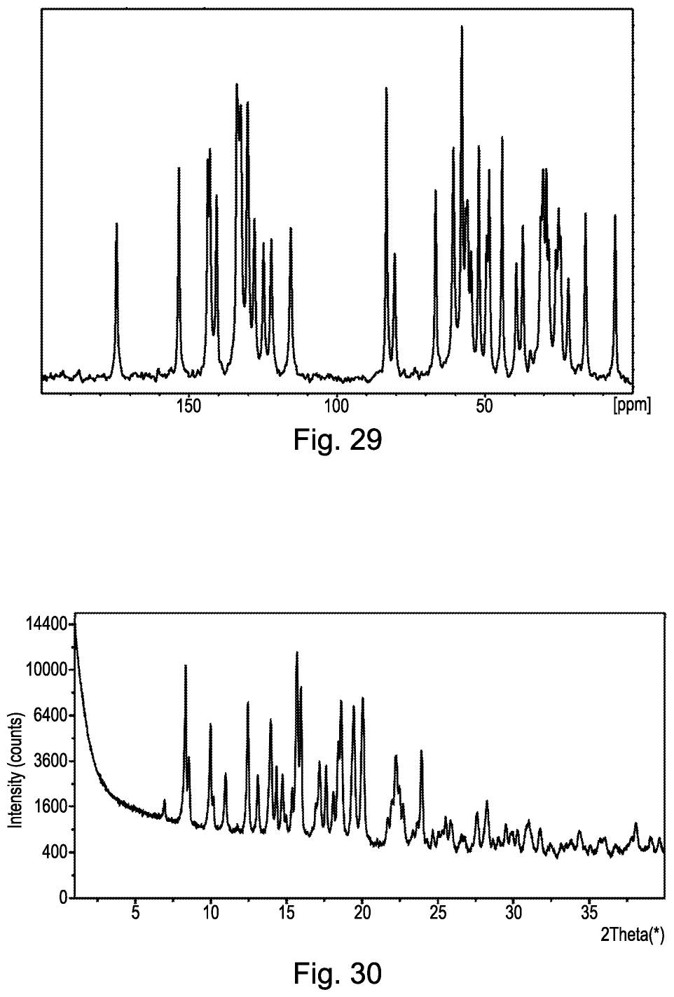

Hydrate Form 5: Hydrate form 5 can be characterized by solid state ¹³C NMR, obtained as set forth in the Examples, having peaks at 5.90, 15.93, 21.71, 24.33, 24.99, 25.92, 28.37, 29.16, 30.25, 31.00, 37.10, 39.31, 44.09, 48.49, 49.30, 51.99, 54.58, 55.81, 56.34, 57.73, 60.59, 66.60, 80.42, 83.22, 115.55, 122.14, 124.75, 127.82, 130.10, 132.40, 133.76, 140.62, 142.89, 143.63, 153.36, and 174.41±0.5 ppm. In some embodiments, hydrate form 5 has a solid state ¹³C NMR substantially as shown in FIG. 29, wherein by “substantially” is meant that the reported peaks can vary by ±0.5 ppm.

Hydrate form 5 can be characterized by an X-ray powder diffraction pattern, obtained as set forth in the Examples, having peaks at 15.8, 16.8, and 19.4±0.2° 2θ using Cu Kα radiation, optionally further characterized by additional peaks at 11.3, 14.5, 18.2, 20.6, and 22.3±0.2° 2θ using Cu Kα radiation, and/or additional peaks at 6.4, 10.7, 12.5, 13.0, 13.5, 16.1, 17.3, 18.6, 19.8, 20.1, 21.8, 24.9, and 26.6±0.2° 2θ using Cu Kα radiation.

In some embodiments, hydrate form 5 has an X-ray powder diffraction pattern substantially as shown in FIG. 26, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

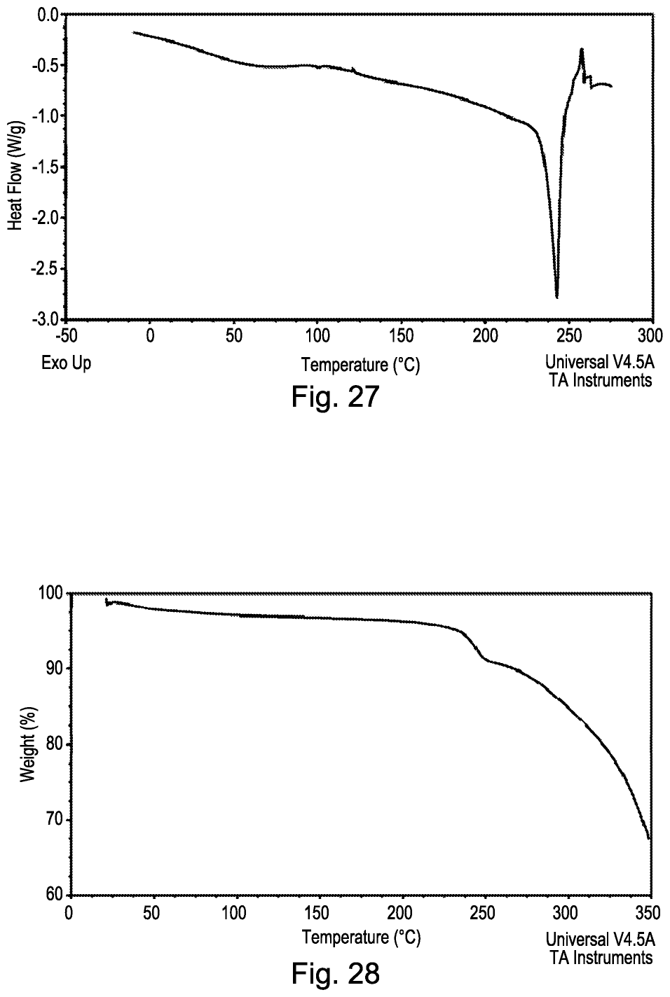

Differential scanning calorimetry (DSC) thermographs were obtained, as set forth in the Examples, for hydrate form 5. The DSC curve indicates an endothermic transition at 237° C.±3° C. Thus, in some embodiments, hydrate form 5 can be characterized by a DSC thermograph having a transition endotherm with an onset of 234° C. to 240° C. For example, in some embodiments hydrate form 5 is characterized by DSC, as shown in FIG. 27.

Hydrate form 5 can be characterized by thermogravimetric analysis (TGA). Thus, hydrate form 5 can be characterized by a weight loss in a range of about 0% to about 2.3% with an onset temperature of about 225° C. In some embodiments, hydrate form 5 has a thermogravimetric analysis substantially as depicted in FIG. 28, wherein by “substantially” is meant that the reported TGA features can vary by ±5° C.

Anhydrous Form 6: Anhydrous form 6 can be characterized by an X-ray powder diffraction pattern, obtained as set forth in the Examples, having peaks at 8.3, 15.7, 16.0, 18.6, and 20.1±0.2° 2θ using Cu Kα radiation, optionally further characterized by additional peaks at 11.0, 12.5, 14.0, 18.4, 19.5, and 23.9±0.2° 2θ using Cu Kα radiation, and/or additional peaks at 8.6, 13.1, 14.3, 14.7, 15.4, 17.2, 17.6, 18.1, 21.9, 22.2, 22.5, 22.7, and 28.2±0.2° 2θ using Cu Kα radiation. In some embodiments, anhydrous form 6 has an X-ray powder diffraction pattern substantially as shown in FIG. 30, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

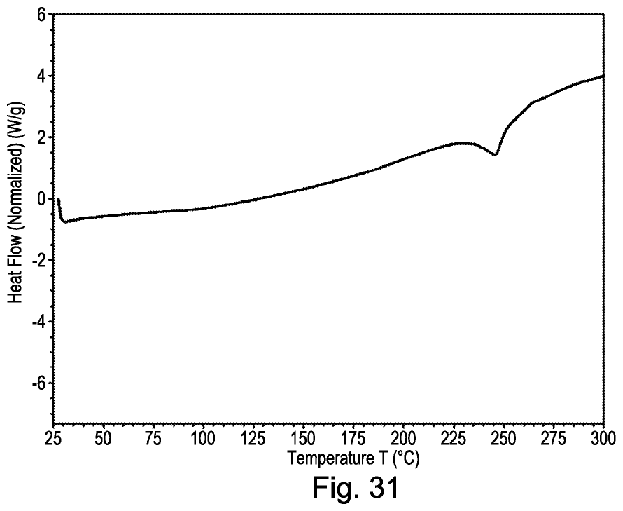

Differential scanning calorimetry (DSC) thermographs were obtained, as set forth in the Examples, for anhydrous form 6. The DSC curve indicates an endothermic transition at 234° C.±3° C. Thus, in some embodiments, anhydrous form 6 can be characterized by a DSC thermograph having a transition endotherm with an onset of 231° C. to 237° C. For example, in some embodiments anhydrous form 6 is characterized by DSC, as shown in FIG. 31.

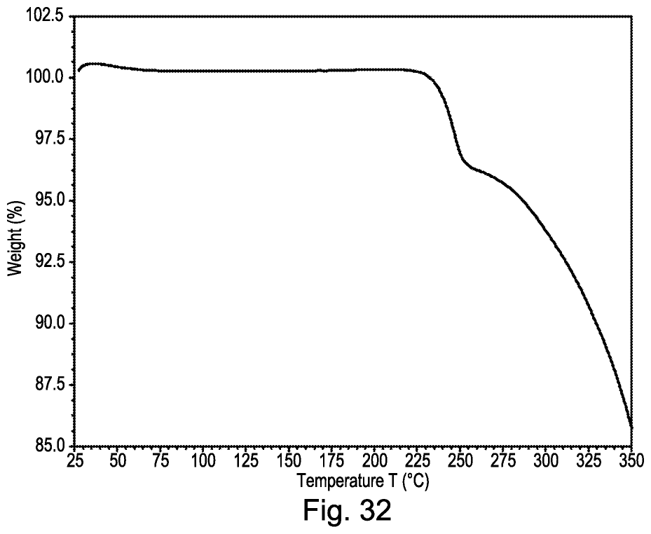

Anhydrous form 6 can be characterized by thermogravimetric analysis (TGA). Thus, anhydrous form 6 can be characterized by a weight loss in a range of about 0% to about 0.3% with an onset temperature of about 25-120° C. In some embodiments, anhydrous form 6 has a thermogravimetric analysis substantially as depicted in FIG. 32, wherein by “substantially” is meant that the reported TGA features can vary by ±5° C.

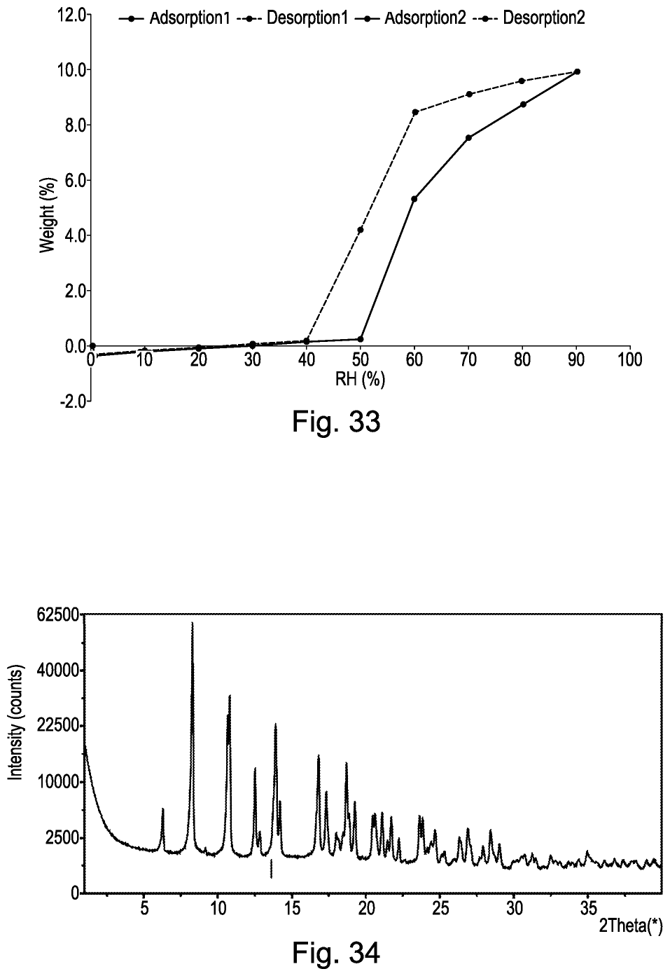

Anhydrous form 6 can be characterized by a moisture sorption profile. For example, in some embodiments, anhydrous form 6 is characterized by the moisture sorption profile as shown in FIG. 33, showing a weight gain of 0.5% from 0-50% RH and 10% 10% by 95% RH.

Hydrate Form 7: Hydrate form 7 can be characterized by an X-ray powder diffraction pattern, obtained as set forth in the Examples, having peaks at 8.3, 10.7, and 10.8±0.2° 2θ using Cu Kα radiation, optionally further characterized by additional peaks at 1.0, 12.5, 13.9, 16.8, 17.3, 18.7, and 19.3±0.2° 2θ using Cu Kα radiation, and/or additional peaks at 6.3, 13.7, 14.2, 16.6, 18.9, 20.5, 20.6, 21.1, 21.7, 23.6, and 23.8 0.2° 2θ using Cu Kα radiation. In some embodiments, hydrate form 7 has an X-ray powder diffraction pattern substantially as shown in FIG. 34, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

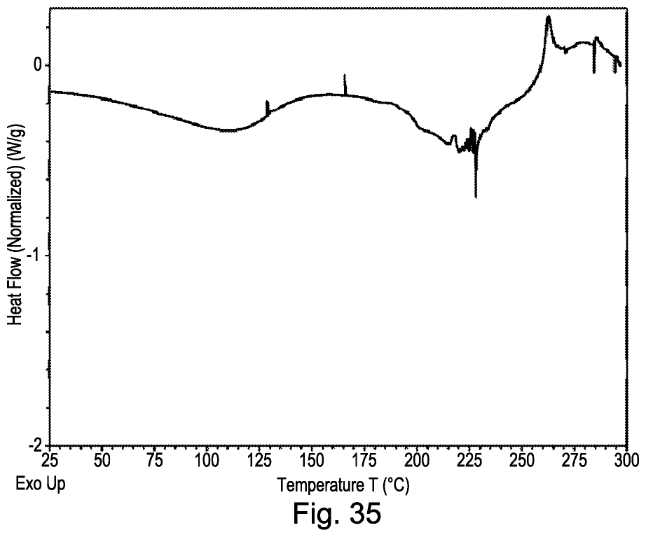

Differential scanning calorimetry (DSC) thermographs were obtained, as set forth in the Examples, for hydrate form 7. The DSC curve indicates an endothermic transition at 220° C.±3° C. Thus, in some embodiments, hydrate form 7 can be characterized by a DSC thermograph having a transition endotherm with an onset of 216° C. to 224° C. For example, in some embodiments hydrate form 7 is characterized by DSC, as shown in FIG. 35.

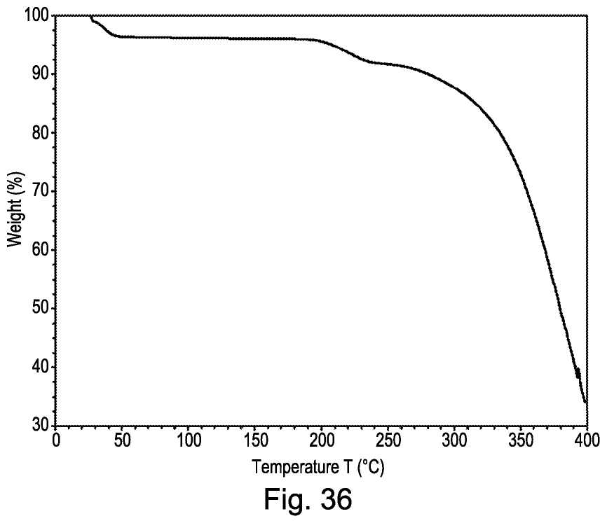

Hydrate form 7 can be characterized by thermogravimetric analysis (TGA). Thus, hydrate form 7 can be characterized by a weight loss in a range of about 0% to about 4.15% with an onset temperature of about 150° C. In some embodiments, hydrate form 7 has a thermogravimetric analysis substantially as depicted in FIG. 36, wherein by “substantially” is meant that the reported TGA features can vary by ±5° C.

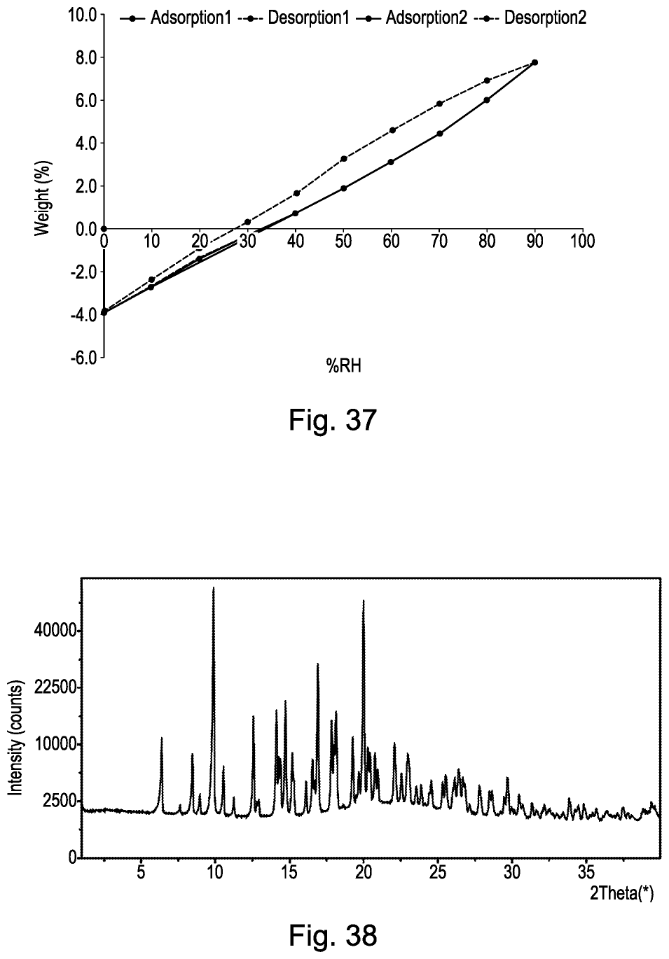

Hydrate form 7 can be characterized by a moisture sorption profile. For example, in some embodiments, hydrate form 7 is characterized by the moisture sorption profile as shown in FIG. 37, showing a weight gain of 0-12% by 95% RH.

Ethanol Solvate Form 8: Ethanol solvate form 8 can be characterized by an X-ray powder diffraction pattern, obtained as set forth in the Examples, having peaks at 9.9, 16.9, and 20.0±0.2° 2θ using Cu Kα radiation, optionally further characterized by additional peaks at 12.6, 14.1, 14.7, 17.8, and 18.1±0.2° 2θ using Cu Kα radiation, and/or additional peaks at 6.4, 8.5, 14.3, 14.4, 15.2, 16.6, 19.3, 20.3, 20.4, 20.8, 22.1, and 23.0±0.2° 2θ using Cu Kα radiation. In some embodiments, ethanol hydrate form 8 has an X-ray powder diffraction pattern substantially as shown in FIG. 38, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

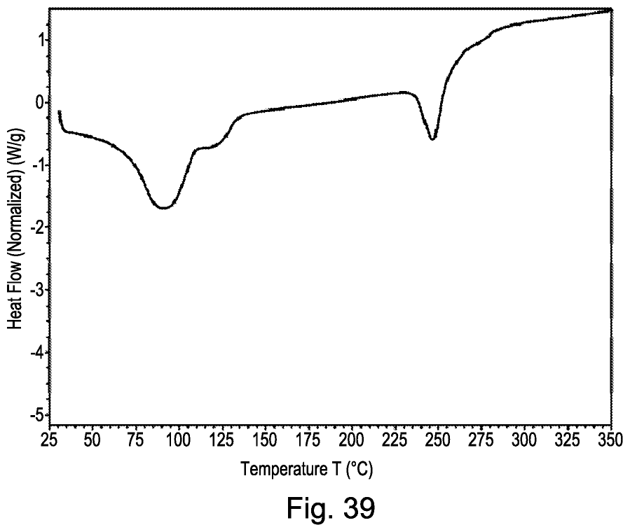

Differential scanning calorimetry (DSC) thermographs were obtained, as set forth in the Examples, for ethanol solvate form 8. The DSC curve indicates an endothermic transition at 67° C. and 236° C.±3° C. Thus, in some embodiments, ethanol solvate form 8 can be characterized by a DSC thermograph having a transition endotherm with an onset of 64° C. to 70° C. and 233° C. to 239° C. For example, in some embodiments ethanol solvate form 8 is characterized by DSC, as shown in FIG. 39.

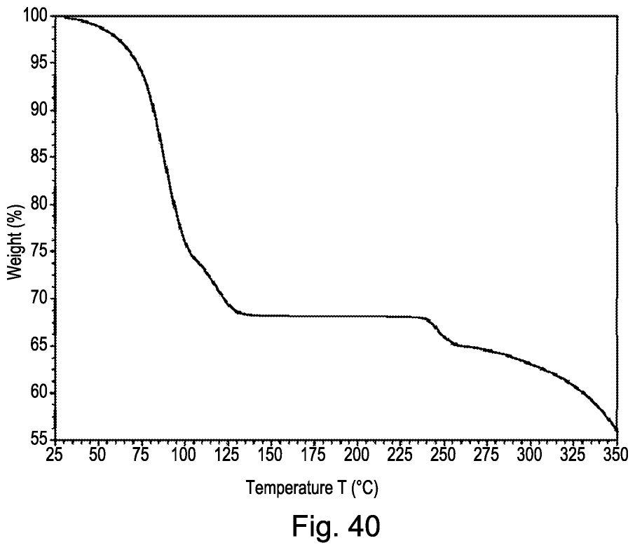

Ethanol solvate form 8 can be characterized by thermogravimetric analysis (TGA). Thus, ethanol solvate form 8 can be characterized by a weight loss in a range of about 0% to about 31.3% with an onset temperature of about 37-140° C. In some embodiments, ethanol solvate form 8 has a thermogravimetric analysis substantially as depicted in FIG. 40, wherein by “substantially” is meant that the reported TGA features can vary by ±5° C.

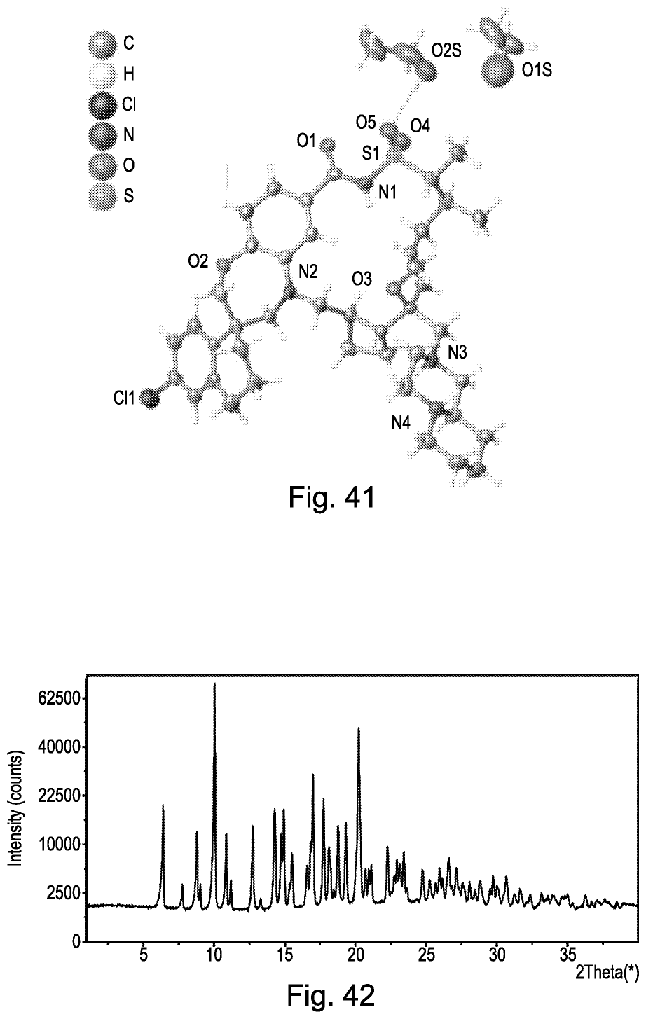

Ethanol solvate form 8 can be characterized by a single crystal structure substantially as shown in FIG. 41, or as set forth in the Examples.

Hydrate Form 9: Hydrate form 9 can be characterized by an X-ray powder diffraction pattern, obtained as set forth in the Examples, having peaks at 10.0, 17.0, and 20.2±0.2° 2θ using Cu Kα radiation, optionally further characterized by additional peaks at 6.4, 14.3, 14.9, 17.8, and 19.3±0.2° 2θ using Cu Kα radiation, and/or additional peaks at 8.8, 10.9, 12.7, 14.8, 15.5, 16.8, 18.1, 18.8, 22.3, and 23.4±0.2° 2θ using Cu Kα radiation. In some embodiments, hydrate form 9 has an X-ray powder diffraction pattern substantially as shown in FIG. 42, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

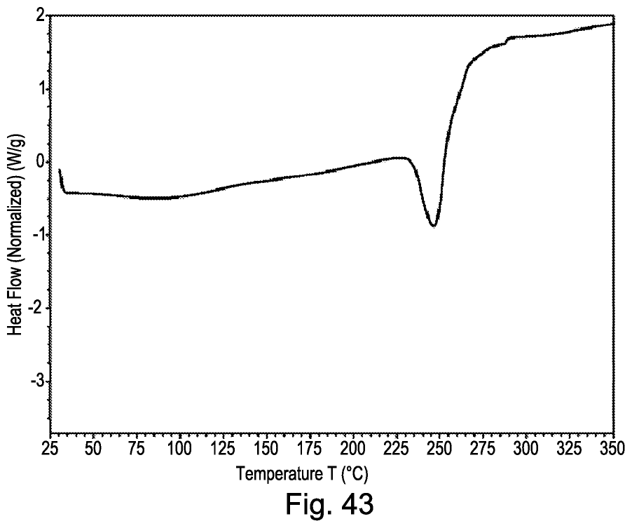

Differential scanning calorimetry (DSC) thermographs were obtained, as set forth in the Examples, for hydrate form 9. The DSC curve indicates an endothermic transition at 234° C.±3° C. Thus, in some embodiments, hydrate form 9 can be characterized by a DSC thermograph having a transition endotherm with an onset of 231° C. to 237° C. For example, in some embodiments hydrate form 7 is characterized by DSC, as shown in FIG. 43.

Hydrate form 9 can be characterized by thermogravimetric analysis (TGA). Thus, hydrate form 9 can be characterized by a weight loss in a range of about 0% to about 1.8% with an onset temperature of about 37-130° C. In some embodiments, hydrate form 9 has a thermogravimetric analysis substantially as depicted in FIG. 44, wherein by “substantially” is meant that the reported TGA features can vary by ±5° C.

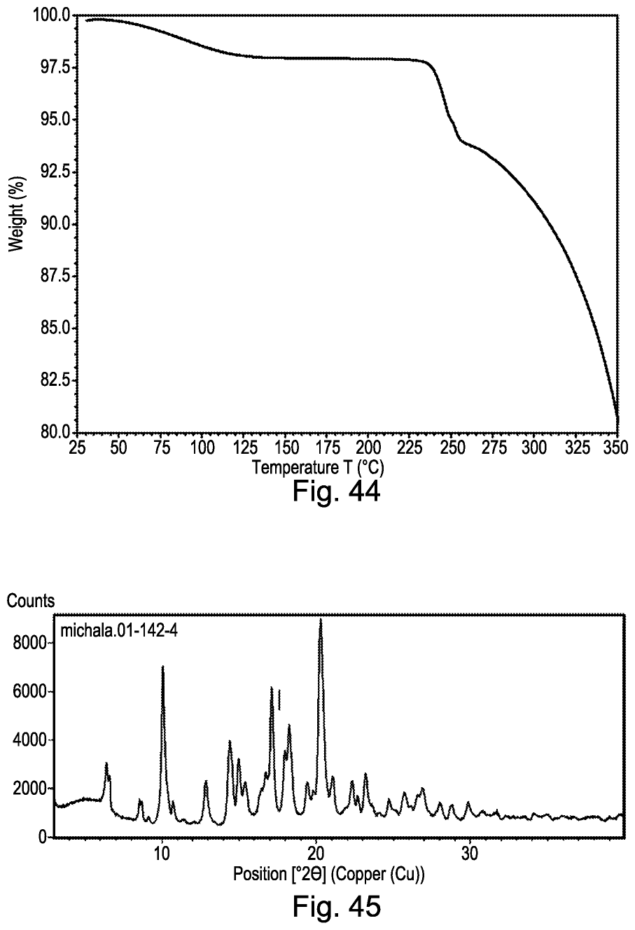

Hydrate Form 10: Hydrate form 10 can be characterized by an X-ray powder diffraction pattern, obtained as set forth in the Examples, having peaks at 10.1, 20.2, and 20.3±0.2° 2θ using Cu Kα radiation, optionally further characterized by additional peaks at 14.4, 14.9, 17.1, 17.9, and 18.3±0.2° 2θ using Cu Kα radiation, and/or additional peaks at 6.4, 6.6, 8.5, 10.7, 12.8, 15.4, 16.3, 16.7, 19.4, 19.8, 21.1, 22.3, 23.2, 25.7, 26.5, and 26.9±0.2° 2θ using Cu Kα radiation. In some embodiments, hydrate form 9 has an X-ray powder diffraction pattern substantially as shown in FIG. 45, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

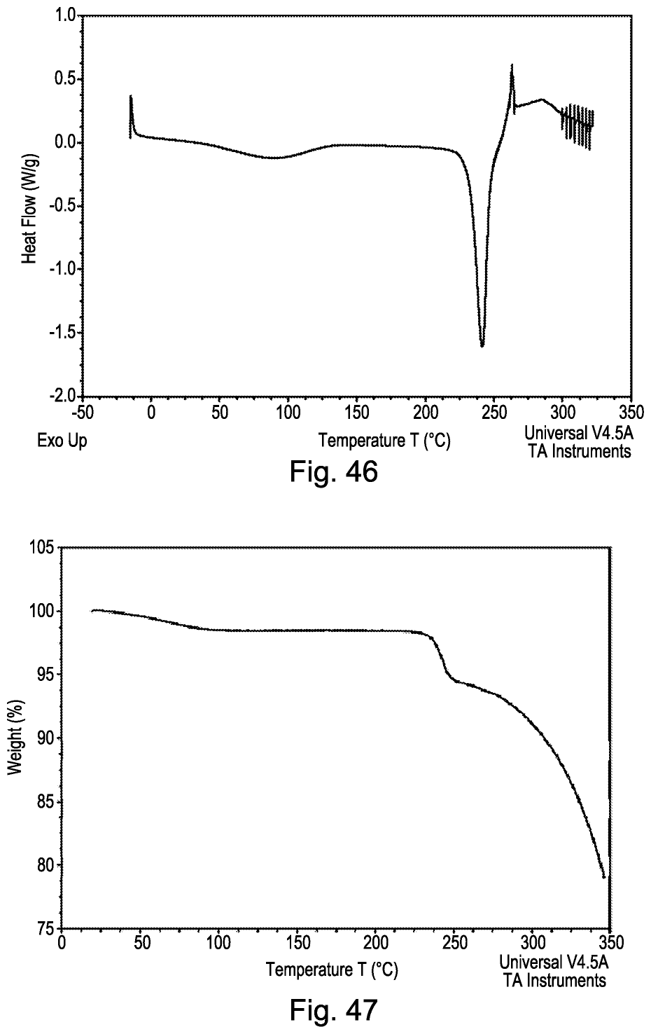

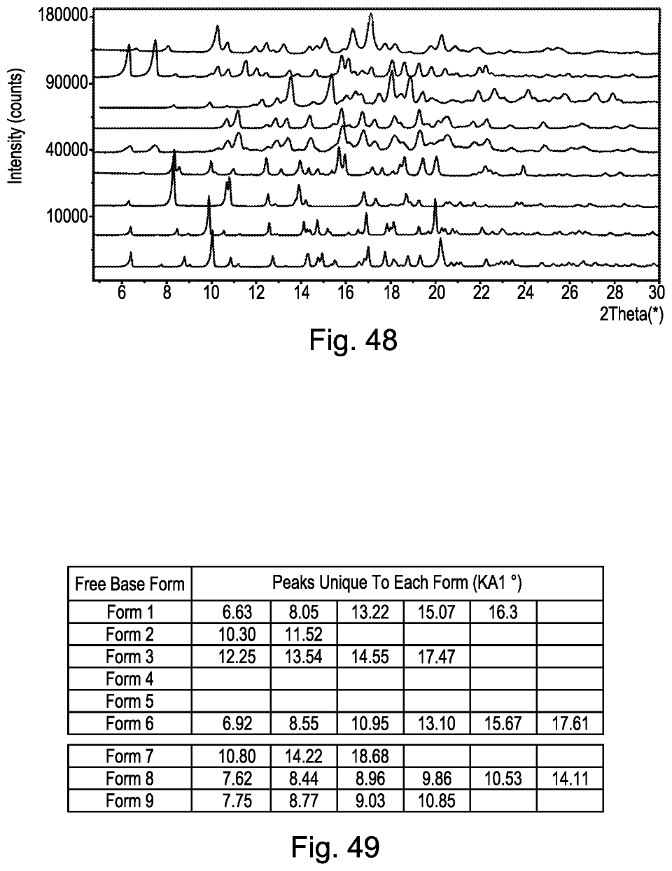

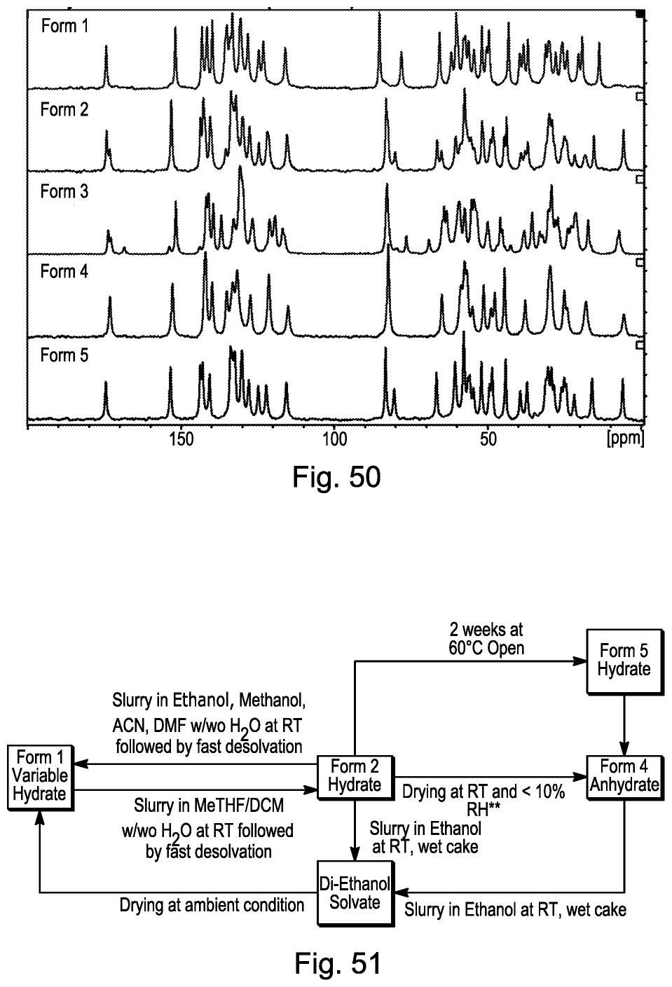

Differential scanning calorimetry (DSC) thermographs were obtained, as set forth in the Examples, for hydrate form 10. The DSC curve indicates an endothermic transition at 233° C.±3° C. Thus, in some embodiments, hydrate form 10 can be characterized by a DSC thermograph having a transition endotherm with an onset of 230° C. to 236° C. For example, in some embodiments hydrate form 7 is characterized by DSC, as shown in FIG. 46.

Hydrate form 10 can be characterized by thermogravimetric analysis (TGA). Thus, hydrate form 10 can be characterized by a weight loss in a range of about 0% to about 1.63% with an onset temperature of about 25-220° C. In some embodiments, hydrate form 10 has a thermogravimetric analysis substantially as depicted in FIG. 47, wherein by “substantially” is meant that the reported TGA features can vary by ±5° C.

### Pharmaceutical Formulations

Provided herein are pharmaceutical formulations comprising a crystalline form as disclosed herein and a pharmaceutically acceptable excipient.

In some embodiments, the pharmaceutical formulation is in the form of a tablet. In some embodiments, the pharmaceutical formulation is in the form of an immediate release tablet. Solid oral drug compositions (e.g., tablets) or preparations have various release profiles, such as an immediate release profile as referenced by FDA guidelines (“Dissolution Testing of Immediate Release Solid Oral Dosage Forms”, issued August 1997, Section IV-A). In the dissolution testing guideline for immediate release profiles, materials which dissolve at least 80% in the first 30 to 60 minutes in solution qualify as immediate release profiles.

Therefore, immediate release solid dosage forms permit the release of most or all of the active ingredient over a short period of time, such as 60 minutes or less, and make rapid absorption of the drug possible. In contrast, extended release solid oral dosage forms permit the release of the active ingredient over an extended period of time in an effort to maintain therapeutically effective plasma levels over similarly extended time intervals, improve dosing compliance, and/or to modify other pharmacokinetic properties of the active ingredient. [00128]“Pharmaceutically acceptable excipient” refers to a broad range of ingredients that may be combined with a compound or salt of the present invention to prepare a pharmaceutical composition or formulation.

Excipients are additives that are included in a formulation because they either impart or enhance the stability, delivery and manufacturability of a drug product, and are physiologically innocuous to the recipient thereof.

Regardless of the reason for their inclusion, excipients are an integral component of a drug product and therefore need to be safe and well tolerated by patients. Given the teachings and guidance provided herein, those skilled in the art will readily be able to vary the amount or range of excipient without increasing viscosity to an undesirable level. Excipients may be chosen to achieve a desired bioavailability, desired stability, resistance to aggregation or degradation or precipitation, protection under conditions of freezing, lyophilization or high temperatures, or other properties. Typically, excipients include, but are not limited to, diluents, colorants, vehicles, anti-adherants, glidants, disintegrants, flavoring agents, coatings, binders, sweeteners, lubricants, sorbents, preservatives, and the like. Examples of suitable excipients are well known to the person skilled in the art of tablet formulation and may be found e.g. in Handbook of Pharmaceutical Excipients (eds. Rowe, Sheskey & Quinn), 6th edition 2009.

As used herein the term “excipients” is intended to refer to inter alia basifying agents, solubilizers, glidants, fillers, binders, lubricant, diluents, preservatives, surface active agents, dispersing agents and the like.

The term also includes agents such as sweetening agents, flavoring agents, coloring agents and preserving agents. Such components will generally be present in admixture within the tablet.

Examples of solubilizers include, but are not limited to, ionic surfactants (including both ionic and non-ionic surfactants) such as sodium lauryl sulfate, cetyltrimethylammonium bromide, polysorbates (such as polysorbate 20 or 80), poloxamers (such as poloxamer 188 or 207), and macrogols.

Examples of lubricants, glidants and flow aids include, but are not limited to, magnesium stearate, calcium stearate, stearic acid, hydrogenated vegetable oil, glyceryl palmitostearate, glyceryl behenate, sodium stearyl fumarate, colloidal silicon dioxide, and talc. The amount of lubricant in a tablet can generally be between 0.1-5% by weight.

Examples of disintegrants include, but are not limited to, starches, celluloses, cross-linked PVP, sodium starch glycolate, croscarmellose sodium, etc.

Examples of fillers (also known as bulking agents or diluents) include, but are not limited to, starches, maltodextrins, polyols (such as lactose), and celluloses. Tablets provided herein may include lactose and/or microcrystalline cellulose. Lactose can be used in anhydrous or hydrated form (e.g. monohydrate), and is typically prepared by spray drying, fluid bed granulation, or roller drying.

Examples of binders include, but are not limited to, cross-linked PVP, HPMC, microcrystalline cellulose, sucrose, starches, etc.

In some embodiments, the pharmaceutically acceptable excipients can comprise one or more diluent, binder, or disintegrant. In embodiments, the pharmaceutically acceptable excipients can comprise a diluent comprising one or more of microcrystalline cellulose, starch, dicalcium phosphate, lactose, sorbitol, mannitol, sucrose, and methyl dextrins, a binder comprising one or more of povidone, hydroxypropyl methylcellulose, hydroxypropyl cellulose, and sodium carboxymethylcellulose, and a disintegrant comprising one or more of crospovidine, sodium starch glycolate, and croscarmellose sodium.

Tablets provided herein may be uncoated or coated (in which case they include a coating). Although uncoated tablets may be used, it is more usual to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water soluble material included in the film coating of the present invention may include a single polymer material, it may also be formed using a mixture of more than one polymer. The coating may be white or colored e.g. gray. Suitable coatings include, but are not limited to, polymeric film coatings such as those comprising polyvinyl alcohol e.g. ‘Opadry® II’ (which includes part-hydrolysed PVA, titanium dioxide, macrogol 3350 and talc, with optional coloring such as iron oxide or indigo carmine or iron oxide yellow or FD&C yellow #6). The amount of coating will generally be between 2-4% of the core's weight, and in certain specific embodiments, 3%. Unless specifically stated otherwise, where the dosage form is coated, it is to be understood that a reference to % weight of the tablet means that of the total tablet, i.e. including the coating.

The pharmaceutical formulations disclosed herein can further comprise a surfactant. As used herein, the surfactant can be cationic, anionic, or non-ionic. In some embodiments, the pharmaceutical formulation can comprise a non-ionic surfactant. In some embodiments, the surfactant can comprise a polysorbate, a poloxamer, or a combination thereof. In some embodiments, the surfactant can comprise polysorbate 20, polysorbate 60, polysorbate 80, or a combination thereof.

### Methods of Treating a Subject

Further provided herein are methods of treating a subject suffering from cancer, comprising administering to the subject a therapeutically effective amount of a crystalline form as disclosed herein, optionally as a pharmaceutical formulation as disclosed herein. In some embodiments, the cancer is multiple myeloma, non-Hodgkin's lymphoma, or acute myeloid leukemia.

### Preparation of Crystalline Forms

The crystalline forms disclosed herein can be prepared by a variety of methods known to those of skill in the art. For example, the crystalline forms can be prepared from amorphous, crude, or another crystalline form of AMG 397. In some embodiments, AMG 397 is combined with a solvent to form a desired crystalline form, for example as discussed in the examples below. In some embodiments, AMG 397 is dissolved in a solvent, or is combined with a solvent to form a slurry. In some embodiments, AMG 397 is combined with a solvent and the solution or slurry thus formed is aged to form the crystalline forms. In some embodiments, the solution or slurry is heated prior to aging or crystal formation.

### EXAMPLES

The following examples are provided for illustration and are not intended to limit the scope of the invention.

### Materials and Methods

Commercially available reagents are used as is without further purification unless specified.

The synthesis of the starting material (AMG 397) for the following methods is disclosed in U.S. Pat. No. 10,300,075. The crystalline forms disclosed herein may be characterized using conventional means, including physical constants and spectral data.

X-Ray Powder Diffraction: XRPD patterns were collected with a PANalytical X'Pert PRO MPD diffractometer or a PANalytical Empyrean diffractometer using an incident beam of Cu radiation produced using an Optix long, fine-focus source. An elliptically graded multilayer mirror was used to focus Cu Kα X-ray radiation through the specimen and onto the detector. Prior to the analysis, a silicon specimen (NIST SRM 640e) was analyzed to verify the observed position of the Si (111) peak is consistent with the NIST-certified position. A specimen of the sample was sandwiched between 3-μm-thick films and analyzed in transmission geometry. A beam-stop, short antiscatter extension, and antiscatter knife edge, were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b or software v. 5.5.

Alternatively, X-ray powder diffraction (XRPD) data were obtained on a PANalytical X'Pert PRO X-ray diffraction system with RTMS detector. Samples were scanned at ambient temperature in a continuous mode from 5 to 45° (2θ) with step size of 0.0334° at a time per step of 50 s at 45 kV and 40 mA with CuKα radiation (1.541874 Å).

XRPD indexing was conducted with proprietary SSCI software, TRIADS™ disclosed in U.S. Pat. No. 8,576,985.

Differential Scanning Calorimetry: Differential scanning calorimetry (DSC) was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, and the weight was accurately recorded. The pan lid was pierced by the instrument and then inserted into the DSC cell for analysis. A weighed aluminum pan configured as the sample pan was placed on the reference side of the cell.

Alternatively, differential scanning calorimetry (DSC) analysis was conducted on a TA Instruments Q and Discovery Series calorimeter at 10° C./min from 25 to 250-350° C. in an aluminum pan under dry nitrogen at 50 ml/min.

Thermal Analysis: Thermal gravimetric analysis (TGA) and TGA/DSC Combo analyses were performed using a Mettler-Toledo TGA/DSC3+ analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen.

Alternatively, thermal gravimetric analysis (TGA) was performed on a TA Instruments Q and Discovery Series analyzer at 10° C./min from ambient temperature to 250-350° C. in a platinum pan under dry nitrogen at 25 ml/min.

Moisture Sorption: Moisture sorption data was collected using a VTI SGA 100 symmetrical vapor sorption analyzer. A sample size of approximately 5-10 mg was used in a platinum pan. Hygroscopicity was evaluated from 5 to 95% RH in increments of 5% RH. Data for adsorption and desorption cycles were collected. Equilibrium criteria were set at 0.001% weight change in 10 minutes with a maximum equilibration time of 180 minutes.

NMR: Solution proton NMR spectra were acquired by Spectral Data Services of Champaign, IL at 25° C. with a Varian UNITYINOVA-400 spectrometer. Samples were dissolved in DMSO-d6. In some cases, the solution NMR spectra were acquired at SSCI with an Agilent DD2-400 spectrometer using deuterated DMSO or methanol. [00154]¹³C SSNMR data was collected on a Bruker DSX spectrometer operating at 600 MHz (¹H). A 4 mm H/F/X spinning probe operating at a spinning frequency of 14 kHz was used for all experiments. CPMAS with TOSS program was used with a recycle delay of 10 s. A 1H 90° pulse of 2.5 μs and ¹³C 180° pulse of 8 μs were used. Decoupling was carried out using a spinal 64 sequence. 4096 transients were acquired for signal averaging. The data was processed with Topspin 3.0 software.

### Example 1: Amorphous AMG 397

Amorphous AMG 397 was prepared by dissolving 1031.06 mg of AMG 397 in 52 mL tetrahydrofuran (THF) and shaking to form a solution. The solution was then spray dried at a spray rate of 2.5 mL/min with an inlet temperature of 63° C., outlet temperature of 50° C., aspirator at 97%, drying air flow at 0.58 kg/min, nozzle air at 7.0 Sl/m, nozzle cool at 20° C. and cyclone cool at 30° C. Product was collected and dried under vacuum oven at 30° C. with −10 bar pressure for 2 days to remove the residual THF.

### Example 2: AMG 397 Hydrate Form 1

AMG 397 Hydrate Form 1 was formed by combining AMG 397 with ˜10 volumes of 95:5 ethanol/water. The solution was heat cycled to 70° C. in sealed vial for 15 min and then cooled to form AMG 397 Hydrate Form 1, which was characterized as shown in the following tables.

TABLE 1
XRPD Data Table

| Pos. [°2θ] | FWHM [°2θ] | d-spacing [Å] | Height [cts] | Rel. Int. [%] |
|---|---|---|---|---|
| 8.08 | 0.13 | 10.95 | 8694.81 | 13.74 |
| 10.28 | 0.13 | 8.6 | 40462.38 | 63.92 |
| 10.72 | 0.13 | 8.25 | 15279.74 | 24.14 |
| 11.98 | 0.15 | 7.39 | 9563.74 | 15.11 |
| 12.48 | 0.15 | 7.1 | 14996.68 | 23.69 |
| 13.25 | 0.18 | 6.68 | 13655.34 | 21.57 |
| 14.38 | 0.15 | 6.16 | 10404.72 | 16.44 |
| 14.69 | 0.15 | 6.03 | 11131.1 | 17.58 |
| 15.11 | 0.2 | 5.87 | 23166.31 | 36.6 |
| 15.9 | 0.13 | 5.58 | 6572.05 | 10.38 |
| 16.3 | 0.2 | 5.44 | 38727.58 | 61.18 |
| 17.13 | 0.23 | 5.18 | 63299.61 | 100 |
| 17.74 | 0.17 | 5 | 15095.93 | 23.85 |
| 18.23 | 0.2 | 4.87 | 14190.89 | 22.42 |
| 19.78 | 0.2 | 4.49 | 11371.71 | 17.96 |
| 20.29 | 0.18 | 4.38 | 28258.72 | 44.64 |
| 20.88 | 0.2 | 4.25 | 11394.74 | 18 |
| 21.69 | 0.1 | 4.1 | 7304.56 | 11.54 |
| 21.92 | 0.18 | 4.06 | 9295.19 | 14.68 |
| 25.01 | 0.17 | 3.56 | 8487.99 | 13.41 |
| 25.44 | 0.15 | 3.5 | 8971.11 | 14.17 |
| 25.62 | 0.23 | 3.48 | 7561.08 | 11.94 |

TABLE 2
Solid State ¹³C NMR Data

| Peak | v(F1) [ppm] | Intensity [abs] | Intensity [rel] |
|---|---|---|---|
| 1 | 174.30 | 3342800.86 | 5.47 |
| 2 | 151.76 | 4875738.84 | 7.98 |
| 3 | 143.08 | 4937517.05 | 8.08 |
| 4 | 141.47 | 4895517.41 | 8.01 |
| 5 | 139.72 | 5473393.72 | 8.95 |
| 6 | 134.99 | 5045623.66 | 8.25 |
| 7 | 133.87 | 4070943.45 | 6.66 |
| 8 | 133.18 | 6027611.34 | 9.86 |
| 9 | 130.53 | 5629472.55 | 9.21 |
| 10 | 128.11 | 4354315.61 | 7.13 |
| 11 | 124.60 | 2996501.88 | 4.90 |
| 12 | 123.10 | 3691109.91 | 6.04 |
| 13 | 115.91 | 3157834.66 | 5.17 |
| 14 | 85.23 | 6108149.53 | 10.00 |
| 15 | 78.05 | 2851707.20 | 4.67 |
| 16 | 65.65 | 4420846.34 | 7.23 |
| 17 | 61.80 | 2795012.02 | 4.57 |
| 18 | 60.23 | 6067426.56 | 9.93 |
| 19 | 57.78 | 3987290.50 | 6.52 |
| 20 | 57.28 | 4156007.39 | 6.80 |
| 21 | 56.15 | 3763019.48 | 6.16 |
| 22 | 54.40 | 3012506.42 | 4.93 |
| 23 | 51.84 | 4997182.81 | 8.17 |
| 24 | 50.30 | 3249618.88 | 5.32 |
| 25 | 49.53 | 4677813.33 | 7.66 |
| 26 | 43.15 | 5294261.23 | 8.67 |
| 27 | 39.48 | 2715242.14 | 4.44 |
| 28 | 38.27 | 3420418.53 | 5.60 |
| 29 | 36.84 | 3868181.97 | 6.33 |
| 30 | 31.05 | 3434460.58 | 5.62 |
| 31 | 30.09 | 3714100.47 | 6.08 |
| 32 | 27.75 | 2815977.80 | 4.61 |
| 33 | 25.54 | 3625318.16 | 5.93 |
| 34 | 24.04 | 2903757.64 | 4.75 |
| 35 | 20.39 | 2695161.47 | 4.41 |
| 36 | 19.13 | 4118642.73 | 6.74 |
| 37 | 13.57 | 3585801.05 | 5.87 |

Single Crystal Data: A dry powder sample of AMG 397 Form 1 form was used for single crystal structure determination. The specimen chosen for data collection was a needle with the approximate dimensions 0.002×0.008×0.025 mm³. The crystal was mounted on a MiTeGen™ mount with mineral oil (STP Oil Treatment). First diffraction patterns showed the crystal to be of marginal quality giving rise to smeared, elongated and split reflections, and diffracting only weakly.

Diffraction data (φ- and ω-scans) were collected at 100K on a Bruker-AXS X8 Kappa diffractometer coupled to a Bruker APEX2 CCD detector using Cu Kα radiation (λ=1.54178 Å) from an/μS microsource. Data reduction was carried out with the program SAINT[1] and semi-empirical absorption correction based on equivalents was performed with the program SADABS[2]. A summary of crystal properties and data/refinement statistics is given in Table 3.

The structure of AMG 397 Form 1 was determined at 100K in the monoclinic chiral space group P21 with one molecule of compound A and 80% of a water molecule in the asymmetric unit.

TABLE 3
X-ray Single Structure Data

| Wavelength | 1.54178 Å | |
|---|---|---|
| Crystal system | Monoclinic | |
| Space group | P2₁ | |
| Unit cell dimensions | a = 10.9544(10) Å | α = 90° |
| | b = 13.6828(9) Å | β = 92.724(6)° |
| | c = 13.4164(9) Å | γ = 90° |
| Volume | 2008.7(3) Å³ | |
| Z | 2 | |

| | |
|---|---|
| Density (calculated) | 1.289 Mg/m³ |
| Absolute structure parameter | −0.008(18) |

### Example 3: AMG 397 Hydrate Form 2

AMG 397 hydrate Form 2 was formed by slurrying 630 mg of AMG 397 in 6.5 mL MeTHF and 6 mL of water (biphasic). The slurry was heated to 78° C. for ˜5 h and then cooled. The material was then filtered and the cake dried on the frit using a vacuum to provide AMG 397 hydrate Form 2, which was characterized as shown in the following tables.

TABLE 4
XRPD Data Table

| Pos. [°2θ] | FWHM [°2θ] | d-spacing [Å] | Height [cts] | Rel. Int. [%] |
|---|---|---|---|---|
| 6.17 | 0.13 | 14.32 | 13770.7 | 1 77.22 |
| 7.36 | 0.13 | 12.01 | 17833.2 | 9 100.00 |
| 10.17 | 0.13 | 8.7 | 3790.04 | 21.25 |
| 10.63 | 0.15 | 8.32 | 2703.53 | 15.16 |
| 11.42 | 0.17 | 7.75 | 5695.76 | 31.94 |
| 11.9 | 0.17 | 7.44 | 2967.75 | 16.64 |
| 13.35 | 0.17 | 6.63 | 2306.23 | 12.93 |
| 14.54 | 0.17 | 6.09 | 2121.85 | 11.9 |
| 15.71 | 0.17 | 5.64 | 8182.67 | 45.88 |
| 16.01 | 0.13 | 5.54 | 6407.94 | 35.93 |
| 16.26 | 0.11 | 5.45 | 2255.42 | 12.65 |
| 16.58 | 0.15 | 5.35 | 2192.74 | 12.3 |
| 17.05 | 0.17 | 5.2 | 3615.4 | 20.27 |
| 17.96 | 0.21 | 4.94 | 5884.73 | 33 |
| 18.49 | 0.19 | 4.8 | 4593.96 | 25.76 |
| 19.17 | 0.17 | 4.63 | 4703.53 | 26.37 |
| 19.72 | 0.19 | 4.5 | 3565.66 | 19.99 |
| 20.31 | 0.19 | 4.37 | 3786.99 | 21.24 |
| 20.86 | 0.13 | 4.26 | 1952.74 | 10.95 |
| 21.82 | 0.17 | 4.07 | 3796.3 | 21.29 |
| 22.14 | 0.13 | 4.02 | 5066.97 | 28.41 |
| 22.43 | 0.07 | 3.96 | 2139.93 | 12 |
| 25.86 | 0.15 | 3.45 | 1810.64 | 10.15 |

TABLE 5
Solid State ¹³C NMR Data

| Peak | v(F1) [ppm] | Intensity [abs] | Intensity [rel] |
|---|---|---|---|
| 1 | 174.17 | 3800981 | 4.74 |
| 2 | 173.18 | 1951029 | 2.43 |
| 3 | 153.09 | 6878902 | 8.57 |
| 4 | 143.54 | 5108126 | 6.37 |
| 5 | 142.61 | 7104043 | 8.85 |
| 6 | 140.41 | 5210609 | 6.49 |
| 7 | 135.32 | 2004425 | 2.5 |
| 8 | 133.6 | 7704824 | 9.6 |
| 9 | 132.04 | 7312391 | 9.11 |
| 10 | 129.92 | 5183342 | 6.46 |
| 11 | 127.61 | 4264137 | 5.31 |
| 12 | 124.57 | 2630715 | 3.28 |
| 13 | 121.81 | 3734339 | 4.65 |
| 14 | 115.39 | 3412330 | 4.25 |
| 15 | 83.01 | 7026289 | 8.75 |
| 16 | 82.55 | 4945741 | 6.16 |
| 17 | 80.11 | 1727021 | 2.15 |
| 18 | 66.4 | 2922826 | 3.64 |
| 19 | 64.99 | 1794251 | 2.24 |
| 20 | 60.39 | 3259503 | 4.06 |
| 21 | 57.5 | 8025559 | 10 |
| 22 | 55.45 | 3169318 | 3.95 |
| 23 | 54.33 | 2073804 | 2.58 |
| 24 | 51.76 | 4778295 | 5.95 |
| 25 | 49.01 | 2939093 | 3.66 |
| 26 | 48.25 | 4143097 | 5.16 |
| 27 | 44.59 | 3194563 | 3.98 |
| 28 | 43.79 | 5163509 | 6.43 |
| 29 | 39.09 | 1608093 | 2 |
| 30 | 37.74 | 1795042 | 2.24 |
| 31 | 36.86 | 2724560 | 3.39 |
| 32 | 29.87 | 5480975 | 6.83 |
| 33 | 28.91 | 4843549 | 6.03 |
| 34 | 24.87 | 3261282 | 4.06 |
| 35 | 24.2 | 2827211 | 3.52 |
| 36 | 21.54 | 1418289 | 1.77 |
| 37 | 18.06 | 1377257 | 1.72 |
| 38 | 15.29 | 3327603 | 4.15 |
| 39 | 5.65 | 3876183 | 4.83 |

TABLE 6
X-ray Single Structure Data

| Crystal system | Primitive Orthorhombic | |
|---|---|---|
| Extinction Symbol | P 2₁ 2₁ 2₁ | |
| Space group | P2₁2₁2₁ (19) | |
| Unit cell dimensions | a = 11.556 Å | α = 90° |
| | b = 13.281 Å | β = 90° |
| | c = 28.765 Å | γ = 90° |

| | |
|---|---|
| Volume | 4414.6 Å³ |
| Chiral Contents? | Chiral |

### Example 4: AMG 397 Hydrate Form 3

AMG 397 hydrate Form 3 was formed by slurrying ˜705 mg of AMG 397 in 7 mL of IPA at 80° C. on a heating block in a sealed vial with stirring at 50 rpm. After cooling to room temperature, the material was heat cycled two times back to 80° C. then allowed to cool back down on the heating block and allowed to settle overnight at room temperature. The sample was reheated as a slurry to 60° C. then immediately cooled back to room temperature, filtered and washed with 1 mL of isopropyl alcohol to provide AMG 397 hydrate Form 3, which was characterized as shown in the following tables.

TABLE 7
XRPD Data Table

| Pos. [°2θ] | FWHM [°2θ] | d-spacing [Å] | Height [cts] | Rel. Int. [%] |
|---|---|---|---|---|
| 9.94 | 0.13 | 8.9 | 970.9 | 14.8 |
| 12.29 | 0.15 | 7.2 | 1299.61 | 19.81 |
| 12.96 | 0.17 | 6.83 | 1457.54 | 22.22 |
| 13.59 | 0.19 | 6.52 | 5414.52 | 82.54 |
| 14.52 | 0.09 | 6.1 | 681.72 | 10.39 |
| 15.4 | 0.19 | 5.75 | 5603.9 | 85.42 |
| 16.04 | 0.11 | 5.53 | 1666.86 | 25.41 |
| 16.45 | 0.11 | 5.39 | 2400.23 | 36.59 |
| 16.77 | 0.17 | 5.29 | 1312.91 | 20.01 |
| 17.46 | 0.11 | 5.08 | 1839.78 | 28.04 |
| 18.08 | 0.22 | 4.91 | 6560.2 | 100 |
| 18.5 | 0.13 | 4.8 | 1642.04 | 25.03 |
| 18.93 | 0.21 | 4.69 | 4982.89 | 75.96 |
| 19.46 | 0.21 | 4.56 | 1946.75 | 29.68 |
| 19.8 | 0.22 | 4.48 | 827.9 | 12.62 |
| 21.91 | 0.15 | 4.06 | 2237.32 | 34.1 |
| 22.6 | 0.17 | 3.93 | 2561.01 | 39.04 |
| 23.1 | 0.15 | 3.85 | 727.04 | 11.08 |
| 24.17 | 0.15 | 3.68 | 2413.88 | 36.8 |
| 25.28 | 0.15 | 3.52 | 749.01 | 11.42 |
| 25.74 | 0.11 | 3.46 | 1506.26 | 22.96 |
| 27.21 | 0.11 | 3.28 | 1580.22 | 24.09 |
| 27.95 | 0.17 | 3.19 | 1743.58 | 26.58 |
| 30.68 | 0.13 | 2.91 | 740.9 | 11.29 |

TABLE 8
Solid State ¹³C NMR Data

| Peak | v(F1) [ppm] | Intensity [abs] | Intensity [rel] |
|---|---|---|---|
| 1 | 173.61 | 10049533 | 2.48 |
| 2 | 172.8 | 6734534 | 1.66 |
| 3 | 151.61 | 23768466 | 5.86 |
| 4 | 141.7 | 25894199 | 6.39 |
| 5 | 140.98 | 27963011 | 6.9 |
| 6 | 139.39 | 22803598 | 5.63 |
| 7 | 136.76 | 17146575 | 4.23 |
| 8 | 132.8 | 15536159 | 3.83 |
| 9 | 130.68 | 40539675 | 10 |
| 10 | 126.62 | 15640220 | 3.86 |
| 11 | 121.1 | 15238969 | 3.76 |
| 12 | 119.24 | 16906552 | 4.17 |
| 13 | 116.84 | 11095321 | 2.74 |
| 14 | 82.72 | 32209889 | 7.95 |
| 15 | 76.48 | 7494130 | 1.85 |
| 16 | 69.06 | 5862090 | 1.45 |
| 17 | 64.14 | 21469963 | 5.3 |
| 18 | 63.31 | 19344581 | 4.77 |
| 19 | 61.66 | 5051676 | 1.25 |
| 20 | 59.23 | 23540938 | 5.81 |
| 21 | 57.5 | 20640152 | 5.09 |
| 22 | 55.23 | 24577865 | 6.06 |
| 23 | 54.43 | 24646185 | 6.08 |
| 24 | 50 | 14330816 | 3.54 |
| 25 | 45.88 | 16254451 | 4.01 |
| 26 | 45.21 | 10536664 | 2.6 |
| 27 | 37.96 | 10004723 | 2.47 |
| 28 | 35.45 | 18391689 | 4.54 |
| 29 | 33 | 9561381 | 2.36 |
| 30 | 32.09 | 8044066 | 1.98 |
| 31 | 30.12 | 19949222 | 4.92 |
| 32 | 29.13 | 31561780 | 7.79 |
| 33 | 27.79 | 14952904 | 3.69 |
| 34 | 27.01 | 16331625 | 4.03 |
| 35 | 23.74 | 11039978 | 2.72 |
| 36 | 22.75 | 12299968 | 3.03 |
| 37 | 21.14 | 18336542 | 4.52 |
| 38 | 17.2 | 14930347 | 3.69 |
| 39 | 7.07 | 10099597 | 2.49 |

### Example 5: AMG 397 Form 4 Anhydrous

AMG 397 Form 4 anhydrous was formed by slurrying ˜2.5 g AMG 397 by azeotropic drying the material with MeTHF solvent in a 150 ml flask. The sample was taken to dryness and then ˜25 mL MeTHF was added to the flask and 1 mL water. The flask was heated to ˜73° C. before filtering, washing with additional solvent and then drying under nitrogen and vacuum for ˜3 days to provide AMG 397 Form 4 anhydrous, which was characterized as shown in the following tables.

TABLE 9
XRPD Data Table

| Pos. [°2θ] | FWHM [°2θ] | d-spacing [Å] | Height [cts] | Rel. Int. [%] |
|---|---|---|---|---|
| 10.71 | 0.15 | 8.26 | 6724.29 | 49.15 |
| 11.21 | 0.18 | 7.89 | 12197.36 | 89.15 |
| 12.52 | 0.17 | 7.07 | 2484.01 | 18.16 |
| 12.89 | 0.2 | 6.87 | 6874.67 | 50.25 |
| 13.39 | 0.18 | 6.61 | 6387.41 | 46.69 |
| 14.43 | 0.18 | 6.14 | 8493.08 | 62.08 |
| 15.44 | 0.12 | 5.74 | 3117.93 | 22.79 |
| 15.84 | 0.18 | 5.59 | 13681.24 | 100 |
| 16.18 | 0.15 | 5.48 | 2128.08 | 15.55 |
| 16.77 | 0.18 | 5.29 | 11695.98 | 85.49 |
| 17.31 | 0.18 | 5.12 | 6015.86 | 43.97 |
| 18.19 | 0.17 | 4.88 | 8813.63 | 64.42 |
| 18.46 | 0.12 | 4.81 | 3733.03 | 27.29 |
| 19.3 | 0.18 | 4.6 | 13279.92 | 97.07 |
| 19.73 | 0.13 | 4.5 | 2598.85 | 19 |
| 20.13 | 0.17 | 4.41 | 5599.07 | 40.93 |
| 20.36 | 0.1 | 4.36 | 6691.63 | 48.91 |
| 20.63 | 0.23 | 4.3 | 6189.95 | 45.24 |
| 21.73 | 0.18 | 4.09 | 4612.97 | 33.72 |
| 22.32 | 0.18 | 3.98 | 6468.65 | 47.28 |
| 23.29 | 0.1 | 3.82 | 2049.12 | 14.98 |
| 23.37 | 0.12 | 3.81 | 2186.08 | 15.98 |
| 24.85 | 0.22 | 3.58 | 4228.58 | 30.91 |
| 26.02 | 0.17 | 3.42 | 1523.5 | 11.14 |
| 26.5 | 0.12 | 3.36 | 3217.63 | 23.52 |
| 26.8 | 0.08 | 3.33 | 1825.28 | 13.34 |
| 28.05 | 0.08 | 3.18 | 1579.06 | 11.54 |

TABLE 10
Solid State ¹³C NMR Data

| Peak | v(F1) [ppm] | Intensity [abs] | Intensity [rel] |
|---|---|---|---|
| 1 | 173.08 | 4193477 | 4.22 |
| 2 | 152.71 | 5613779 | 5.65 |
| 3 | 141.92 | 9110565 | 9.17 |
| 4 | 139.77 | 5784194 | 5.82 |
| 5 | 135.02 | 4788589 | 4.82 |
| 6 | 133.04 | 5730517 | 5.77 |
| 7 | 131.61 | 7068963 | 7.12 |
| 8 | 127.31 | 4350116 | 4.38 |
| 9 | 121.3 | 6633485 | 6.68 |
| 10 | 114.99 | 3196611 | 3.22 |
| 11 | 82.34 | 9933206 | 10 |
| 12 | 64.86 | 4451853 | 4.48 |
| 13 | 58.58 | 5450602 | 5.49 |
| 14 | 57.48 | 8087075 | 8.14 |
| 15 | 56.72 | 7044327 | 7.09 |
| 16 | 54.92 | 3074377 | 3.1 |
| 17 | 51.26 | 5439714 | 5.48 |
| 18 | 48.86 | 2930052 | 2.95 |
| 19 | 47.61 | 4619419 | 4.65 |
| 20 | 44.44 | 7323821 | 7.38 |
| 21 | 37.7 | 3766570 | 3.79 |
| 22 | 29.56 | 7543149 | 7.59 |
| 23 | 24.95 | 4902202 | 4.94 |
| 24 | 24.02 | 2990135 | 3.01 |
| 25 | 17.86 | 3606095 | 3.63 |
| 26 | 5.55 | 2234979 | 2.25 |

TABLE 11
X-ray Single Structure Data

| Crystal system | Primitive Orthorhombic | |
|---|---|---|
| Extinction Symbol | P 2₁ 2₁ 2₁ | |
| Space group | P2₁2₁2₁ (19) | |
| Unit cell dimensions | a = 10.535 Å | α = 90° |
| | b = 13.767 Å | β = 90° |
| | c = 23.818 Å | γ = 90° |

| | |
|---|---|
| Volume | 4034.5 Å³ |
| Chiral Contents? | Chiral |

### Example 6: AMG 397 Hydrate Form 5

AMG 397 hydrate Form 5 was formed by slurrying ˜20 g of AMG 397 in 8 volumes of MeTHF in a 500 mL reactor and heated jacket to 70° C. To this was added 1 volume (20 mL water) and the slurry mostly dissolved before crystalline material quickly began to come back out of solution. After ˜20 minutes addition of 40 mL heptane began over ˜20 minutes. The slurry was then cooled to 20° C. and agitated slowly. After ˜3 hours an additional 20 mL of heptane was added, and the slurry was simultaneously heated back to 70° C. for ˜45 minutes before cooling back to 20° C. and allowed to age overnight. The material was then filtered and washed with 100 mL of 70:30 MeTHF/heptane, and dried on frit with vacuum and air for ˜6 hours.

TABLE 12
XRPD Data Table

| Pos. [°2θ] | FWHM [°2θ] | d-spacing [Å] | Height [cts] | Rel. Int. [%] |
|---|---|---|---|---|
| 6.4 | 0.18 | 13.8 | 1284.39 | 21.75 |
| 7.59 | 0.17 | 11.64 | 1139.91 | 19.3 |
| 10.28 | 0.17 | 8.6 | 951.67 | 16.11 |
| 10.73 | 0.15 | 8.25 | 2615.41 | 44.29 |
| 11.29 | 0.27 | 7.84 | 3828.9 | 64.84 |
| 11.57 | 0.1 | 7.65 | 941.51 | 15.94 |
| 12.05 | 0.23 | 7.34 | 656.45 | 11.12 |
| 12.52 | 0.17 | 7.07 | 1260.1 | 21.34 |
| 12.97 | 0.08 | 6.82 | 2468.87 | 41.81 |
| 13.48 | 0.17 | 6.57 | 2723.58 | 46.12 |
| 14.47 | 0.2 | 6.12 | 3154.18 | 53.41 |
| 15.83 | 0.23 | 5.6 | 5905.57 | 100 |
| 16.14 | 0.13 | 5.49 | 2173.03 | 36.8 |
| 16.79 | 0.25 | 5.28 | 4945.45 | 83.74 |
| 17.33 | 0.13 | 5.12 | 2376.82 | 40.25 |
| 18.2 | 0.18 | 4.87 | 3832.71 | 64.9 |
| 18.58 | 0.13 | 4.78 | 2206.54 | 37.36 |
| 19.4 | 0.25 | 4.58 | 4199.23 | 71.11 |
| 19.78 | 0.17 | 4.49 | 2100.01 | 35.56 |
| 20.12 | 0.17 | 4.41 | 2485.72 | 42.09 |
| 20.63 | 0.28 | 4.3 | 3490.49 | 59.11 |
| 21.76 | 0.2 | 4.08 | 2307.49 | 39.07 |
| 22.29 | 0.12 | 3.99 | 2958.03 | 50.09 |
| 23.33 | 0.12 | 3.81 | 963.18 | 16.31 |
| 24.91 | 0.27 | 3.57 | 1579.58 | 26.75 |
| 26.04 | 0.23 | 3.42 | 993.04 | 16.82 |
| 26.63 | 0.33 | 3.35 | 1248.69 | 21.14 |
| 28.12 | 0.2 | 3.17 | 629.62 | 10.66 |
| 28.78 | 0.27 | 3.1 | 893.39 | 15.13 |

TABLE 13
Solid State ¹³C NMR Data

| Peak | v(F1) [ppm] | Intensity [abs] | Intensity [rel] |
|---|---|---|---|
| 1 | 174.41 | 2536581 | 4.26 |
| 2 | 153.36 | 3494516 | 5.87 |
| 3 | 143.63 | 3642869 | 6.12 |
| 4 | 142.89 | 3830041 | 6.43 |
| 5 | 140.62 | 3031375 | 5.09 |
| 6 | 133.76 | 4950008 | 8.31 |
| 7 | 132.4 | 4592861 | 7.71 |
| 8 | 130.1 | 4634637 | 7.78 |
| 9 | 127.82 | 2618844 | 4.4 |
| 10 | 124.75 | 2188952 | 3.68 |
| 11 | 122.14 | 2269538 | 3.81 |
| 12 | 115.55 | 2458481 | 4.13 |
| 13 | 83.22 | 4885649 | 8.2 |
| 14 | 80.42 | 2008221 | 3.37 |
| 15 | 66.6 | 3112469 | 5.23 |
| 16 | 60.59 | 3844730 | 6.46 |
| 17 | 57.73 | 5952634 | 10 |
| 18 | 56.34 | 2798490 | 4.7 |
| 19 | 55.81 | 2943539 | 4.94 |
| 20 | 54.58 | 2058953 | 3.46 |
| 21 | 51.99 | 3880193 | 6.52 |
| 22 | 49.3 | 2328414 | 3.91 |
| 23 | 48.49 | 3463168 | 5.82 |
| 24 | 44.09 | 4038022 | 6.78 |
| 25 | 39.31 | 1847964 | 3.1 |
| 26 | 37.1 | 2496021 | 4.19 |
| 27 | 31 | 2667016 | 4.48 |
| 28 | 30.25 | 3474256 | 5.83 |
| 29 | 29.16 | 3478037 | 5.84 |
| 30 | 28.37 | 2263036 | 3.8 |
| 31 | 25.92 | 2083175 | 3.5 |
| 32 | 24.99 | 2796131 | 4.7 |
| 33 | 24.33 | 2310642 | 3.88 |
| 34 | 21.71 | 1584397 | 2.66 |
| 35 | 15.93 | 2710099 | 4.55 |
| 36 | 5.9 | 2677404 | 4.5 |

### Example 7: AMG 397 Form 6 Anhydrous

AMG 397 Form 6 anhydrous was prepared by charging ˜1 g of AMG 397 with 1-propanol and slurrying at 55° C. for 3 days. The isolated solids were subsequently dried under vacuum at 106-108° C. for 3 days.

TABLE 14
XRPD Data Table

| Pos. [°2θ] | FWHM [°2θ] | d-spacing [Å] | Height [cts] | Rel. Int. [%] |
|---|---|---|---|---|
| 6.95 | 0.07 | 12.73 | 624.75 | 5.74 |
| 8.34 | 0.07 | 10.6 | 9022.72 | 82.85 |
| 8.56 | 0.08 | 10.33 | 2744.06 | 25.2 |
| 9.99 | 0.07 | 8.86 | 4867.34 | 44.69 |
| 10.17 | 0.05 | 8.69 | 975.74 | 8.96 |
| 10.98 | 0.1 | 8.06 | 2034.16 | 18.68 |
| 11.76 | 0.07 | 7.52 | 111.66 | 1.03 |
| 12.45 | 0.1 | 7.11 | 6315.74 | 57.99 |
| 13.12 | 0.1 | 6.75 | 2028.32 | 18.62 |
| 13.96 | 0.12 | 6.34 | 5225.42 | 47.98 |
| 14.34 | 0.08 | 6.18 | 2469.23 | 22.67 |
| 14.74 | 0.12 | 6.01 | 2156.02 | 19.8 |
| 14.98 | 0.07 | 5.92 | 507.71 | 4.66 |
| 15.37 | 0.08 | 5.77 | 1525.68 | 14.01 |
| 15.69 | 0.1 | 5.65 | 10890.96 | 100 |
| 15.96 | 0.08 | 5.55 | 7780.96 | 71.44 |
| 16.93 | 0.07 | 5.24 | 995.53 | 9.14 |
| 17.18 | 0.12 | 5.16 | 2869.57 | 26.35 |
| 17.62 | 0.08 | 5.03 | 2702.7 | 24.82 |
| 18.11 | 0.1 | 4.9 | 1515.76 | 13.92 |
| 18.41 | 0.08 | 4.82 | 3976.58 | 36.51 |
| 18.62 | 0.12 | 4.77 | 6853.37 | 62.93 |
| 19.45 | 0.1 | 4.56 | 6444.7 | 59.17 |
| 20.06 | 0.17 | 4.43 | 7023.76 | 64.49 |
| 20.99 | 0.05 | 4.23 | 81.23 | 0.75 |
| 21.67 | 0.12 | 4.1 | 591.52 | 5.43 |
| 21.91 | 0.12 | 4.06 | 1111.11 | 10.2 |
| 22.19 | 0.15 | 4.01 | 3262.39 | 29.96 |
| 22.47 | 0.1 | 3.96 | 1899.39 | 17.44 |
| 22.72 | 0.1 | 3.91 | 1135.54 | 10.43 |
| 23.37 | 0.1 | 3.81 | 292.05 | 2.68 |
| 23.61 | 0.1 | 3.77 | 555 | 5.1 |
| 23.92 | 0.12 | 3.72 | 3702.69 | 34 |
| 24.28 | 0.1 | 3.67 | 132.56 | 1.22 |
| 24.68 | 0.1 | 3.61 | 365.49 | 3.36 |
| 25.05 | 0.08 | 3.55 | 307.45 | 2.82 |
| 25.29 | 0.07 | 3.52 | 369.87 | 3.4 |
| 25.51 | 0.12 | 3.49 | 751.21 | 6.9 |
| 25.81 | 0.1 | 3.45 | 561.98 | 5.16 |
| 25.95 | 0.08 | 3.43 | 442.95 | 4.07 |
| 26.56 | 0.05 | 3.36 | 224.42 | 2.06 |
| 26.82 | 0.12 | 3.32 | 175.57 | 1.61 |
| 27.26 | 0.05 | 3.27 | 80.06 | 0.74 |
| 27.55 | 0.08 | 3.24 | 812.14 | 7.46 |
| 27.63 | 0.07 | 3.23 | 885.69 | 8.13 |
| 28.23 | 0.15 | 3.16 | 1275.56 | 11.71 |
| 28.66 | 0.08 | 3.12 | 187.9 | 1.73 |
| 29.02 | 0.17 | 3.08 | 209.02 | 1.92 |
| 29.52 | 0.13 | 3.03 | 551.11 | 5.06 |
| 29.77 | 0.08 | 3 | 297.93 | 2.74 |
| 29.96 | 0.08 | 2.98 | 358.11 | 3.29 |
| 30.31 | 0.13 | 2.95 | 396.01 | 3.64 |
| 30.82 | 0.13 | 2.9 | 463.4 | 4.25 |
| 31.03 | 0.07 | 2.88 | 654.11 | 6.01 |
| 31.71 | 0.14 | 2.82 | 401.12 | 3.68 |
| 31.84 | 0.12 | 2.82 | 315.98 | 2.9 |
| 32.41 | 0.08 | 2.76 | 96.48 | 0.89 |
| 33.13 | 0.06 | 2.7 | 107.94 | 0.99 |
| 33.49 | 0.12 | 2.67 | 101.69 | 0.93 |
| 33.8 | 0.24 | 2.65 | 179.17 | 1.65 |
| 34.32 | 0.24 | 2.61 | 397.13 | 3.65 |
| 35.06 | 0.1 | 2.56 | 114.87 | 1.05 |
| 35.62 | 0.08 | 2.52 | 178.18 | 1.64 |
| 36.07 | 0.1 | 2.49 | 247.36 | 2.27 |
| 36.76 | 0.06 | 2.44 | 137.02 | 1.26 |
| 37.79 | 0.12 | 2.38 | 267.98 | 2.46 |
| 38.09 | 0.18 | 2.36 | 641.09 | 5.89 |
| 39.04 | 0.29 | 2.31 | 253.52 | 2.33 |
| 39.64 | 0.16 | 2.27 | 251.73 | 2.31 |

### ¹H NMR Data

¹H NMR (400 MHz, DMSO-d6) δ ppm 0.91 (br d, J=6.61 Hz, 3H) 1.18 (br s, 2H) 1.21-1.29 (m, 1H) 1.30-1.47 (m, 3H) 1.48-1.72 (m, 4H) 1.73-1.88 (m, 2H) 2.00 (br d, J=13.85 Hz, 2H) 2.12-2.29 (m, 3H) 2.29-2.39 (m, 1H) 2.39-2.48 (m, 2H) 2.54-2.82 (m, 4H) 2.86-3.01 (m, 2H) 3.01-3.16 (m, 1H) 3.17-3.26 (m, 3H) 3.46-3.66 (m, 3H) 3.66-3.79 (m, 2H) 3.86 (br d, J=14.28 Hz, 2H) 3.91-4.08 (m, 3H) 5.23-5.52 (m, 2H) 5.61 (br d, J=15.77 Hz, 1H) 6.81 (br d, J=8.10 Hz, 1H) 6.91-7.12 (m, 2H) 7.17 (d, J=2.13 Hz, 2H) 7.27 (dd, J=8.42, 2.24 Hz, 1H) 7.67 (d, J=8.52 Hz, 1H).

TABLE 15
X-ray Single Structure Data

| Crystal system | Primitive Orthorhombic | |
|---|---|---|
| Extinction Symbol | P 2₁ 2₁ 2₁ | |
| Space group | P2₁2₁2₁ (19) | |
| Unit cell dimensions | a = 12.341 Å | α = 90° |
| | b = 16.116 Å | β = 90° |
| | c = 20.623 Å | γ = 90° |

| | |
|---|---|
| Volume | 4101.8 Å³ |
| Chiral Contents? | Chiral |

### Example 8: AMG 397 Hydrate Form 7

AMG 397 hydrate Form 7 was prepared by charging ˜4 g of AMG 397 with 1-propanol and then slurrying at 55° C. for 4 days. Isolated solids were subsequently dried under vacuum at 95-105° C. for 7 days. Water content by KF was 0.9% initially and 5.6% after equilibration at ambient conditions. DVS indicates variable water content to 0-12% based on environment.

TABLE 16
XRPD Data Table

| Pos. [°2θ] | FWHM [°2θ] | d-spacing [Å] | Height [cts] | Rel. Int. [%] |
|---|---|---|---|---|
| 1.01 | 0.05 | 87.15 | 14412.15 | 25.28 |
| 6.3 | 0.08 | 14.02 | 4275.95 | 7.5 |
| 8.3 | 0.07 | 10.65 | 57008.04 | 100 |
| 9.17 | 0.08 | 9.65 | 398.48 | 0.7 |
| 10.67 | 0.07 | 8.29 | 24233.22 | 42.51 |
| 10.81 | 0.07 | 8.18 | 30451.54 | 53.42 |
| 12.52 | 0.08 | 7.07 | 11377.02 | 19.96 |
| 12.86 | 0.1 | 6.89 | 1959.12 | 3.44 |
| 13.74 | 0.05 | 6.45 | 5326.33 | 9.34 |
| 13.9 | 0.12 | 6.37 | 21684.22 | 38.04 |
| 14.21 | 0.1 | 6.23 | 5619.66 | 9.86 |
| 16.64 | 0.03 | 5.33 | 3573.2 | 6.27 |
| 16.82 | 0.12 | 5.27 | 14167.88 | 24.85 |
| 17.33 | 0.1 | 5.12 | 7265.15 | 12.74 |
| 17.99 | 0.08 | 4.93 | 1995.29 | 3.5 |
| 18.16 | 0.07 | 4.88 | 1529.53 | 2.68 |
| 18.46 | 0.1 | 4.81 | 2028.12 | 3.56 |
| 18.69 | 0.1 | 4.75 | 12727.92 | 22.33 |
| 18.89 | 0.07 | 4.7 | 4101.51 | 7.19 |
| 19.27 | 0.12 | 4.61 | 5856.36 | 10.27 |
| 19.79 | 0.07 | 4.49 | 222.13 | 0.39 |
| 20.46 | 0.08 | 4.34 | 4002.26 | 7.02 |
| 20.63 | 0.08 | 4.3 | 4209.42 | 7.38 |
| 21.11 | 0.12 | 4.21 | 4279.73 | 7.51 |
| 21.46 | 0.08 | 4.14 | 1360.5 | 2.39 |
| 21.72 | 0.1 | 4.09 | 3885.97 | 6.82 |
| 22.24 | 0.1 | 4 | 1616.44 | 2.84 |
| 22.63 | 0.1 | 3.93 | 131.88 | 0.23 |
| 23.63 | 0.1 | 3.77 | 4067.87 | 7.14 |
| 23.84 | 0.1 | 3.73 | 3739.66 | 6.56 |
| 24.16 | 0.08 | 3.68 | 972.63 | 1.71 |
| 24.33 | 0.06 | 3.65 | 1115.84 | 1.96 |
| 24.42 | 0.07 | 3.64 | 1410.53 | 2.47 |
| 24.69 | 0.13 | 3.61 | 2476.43 | 4.34 |
| 25.08 | 0.07 | 3.55 | 421.15 | 0.74 |
| 25.32 | 0.12 | 3.52 | 762.51 | 1.34 |
| 25.99 | 0.13 | 3.43 | 314.23 | 0.55 |
| 26.31 | 0.12 | 3.39 | 1772.13 | 3.11 |
| 26.44 | 0.08 | 3.37 | 1411.28 | 2.48 |
| 26.89 | 0.12 | 3.32 | 2712.01 | 4.76 |
| 27.1 | 0.07 | 3.29 | 1222.33 | 2.14 |
| 27.69 | 0.07 | 3.22 | 481.75 | 0.85 |
| 27.92 | 0.13 | 3.2 | 1150.49 | 2.02 |
| 28.41 | 0.07 | 3.14 | 2488.03 | 4.36 |
| 28.74 | 0.1 | 3.11 | 545 | 0.96 |
| 29.02 | 0.13 | 3.08 | 1409.22 | 2.47 |
| 29.96 | 0.17 | 2.98 | 303.43 | 0.53 |
| 30.21 | 0.1 | 2.96 | 355.82 | 0.62 |
| 30.57 | 0.12 | 2.92 | 549.24 | 0.96 |
| 30.78 | 0.17 | 2.9 | 585.07 | 1.03 |
| 31.24 | 0.08 | 2.86 | 773.08 | 1.36 |
| 31.46 | 0.08 | 2.84 | 438.4 | 0.77 |
| 31.98 | 0.17 | 2.8 | 32.02 | 0.06 |
| 32.47 | 0.12 | 2.76 | 589.55 | 1.03 |
| 32.83 | 0.1 | 2.73 | 332.33 | 0.58 |
| 33.13 | 0.13 | 2.7 | 285.27 | 0.5 |
| 33.68 | 0.07 | 2.66 | 261.11 | 0.46 |
| 33.96 | 0.1 | 2.64 | 273.38 | 0.48 |
| 34.41 | 0.15 | 2.61 | 371.08 | 0.65 |
| 34.96 | 0.05 | 2.57 | 875.99 | 1.54 |
| 35.69 | 0.1 | 2.52 | 192.57 | 0.34 |
| 36.09 | 0.1 | 2.49 | 305.44 | 0.54 |
| 36.8 | 0.08 | 2.44 | 427.9 | 0.75 |
| 37.35 | 0.08 | 2.41 | 305.31 | 0.54 |
| 37.94 | 0.13 | 2.37 | 309.9 | 0.54 |
| 38.24 | 0.07 | 2.35 | 348.42 | 0.61 |
| 38.92 | 0.07 | 2.31 | 259.82 | 0.46 |
| 39.42 | 0.17 | 2.29 | 346.58 | 0.61 |

### ¹H NMR Data

¹H NMR (400 MHz, DMSO-d6) δ ppm 0.91 (br d, J=6.61 Hz, 2H) 1.17 (br d, J=6.61 Hz, 3H) 1.21-1.29 (m, 1H) 1.29-1.49 (m, 3H) 1.49-1.70 (m, 4H) 1.70-1.91 (m, 3H) 1.92-2.11 (m, 2H) 2.11-2.37 (m, 4H) 2.39-2.48 (m, 2H) 2.53-2.82 (m, 4H) 2.86-3.01 (m, 2H) 3.01-3.17 (m, 2H) 3.17-3.26 (m, 3H) 3.42-3.64 (m, 2H) 3.72 (br s, 2H) 3.78-3.91 (m, 2H) 3.91-4.07 (m, 2H) 5.32-5.52 (m, 1H) 5.52-5.77 (m, 2H) 6.81 (br d, J=8.10 Hz, 2H) 6.94-7.13 (m, 1H) 7.17 (d, J=2.34 Hz, 2H) 7.26 (dd, J=8.52, 2.13 Hz, 1H) 7.67 (d, J=8.74 Hz, 1H).

TABLE 17
X-ray Single Structure Data

| Crystal system | Primitive Monoclinic | |
|---|---|---|
| Extinction Symbol | P 1 2₁ 1 | |
| Space group | P2₁(4) | |
| Unit cell dimensions | a = 10.226 Å | α = 90° |
| | b = 16.351 Å | β = 109.84° |
| | c = 14.888 Å | γ = 90° |

| | |
|---|---|
| Volume | 2314.6 Å³ |
| Chiral Contents? | Chiral |

### Example 9: AMG 397 Ethanol Solvate Form 8

AMG 397 ethanol solvate Form 8 was prepared by charging AMG 397 with EtOH and stirring at 55° C. for 3 days.

For single crystal AMG 397 ethanol solvate was formed by dissolving ˜500 mg AMG 397 in 5 mL ethanol and 1.5 eq of 5N NaOH. The sample was heated on a hot plate to 60° C. Then 0.75 eq 6M acetic acid was added to the solution with stirring. The solution was then allowed to age at 60° C. without stirring. An additional 0.25 eq 6M acetic acid was added and the sample aged overnight in a sealed vial. The vial was cooled to 50° C. and held overnight, then 40° C. and held overnight. The sample was then cooled to 30° C. as crystals had precipitated.

TABLE 18
XRPD Data Table

| Pos. [°2θ] | FWHM [°2θ] | d-spacing [Å] | Height [cts] | Rel. Int. [%] |
|---|---|---|---|---|
| 2.67 | 0.4 | 33.12 | 97.19 | 0.18 |
| 6.39 | 0.05 | 13.83 | 9303.59 | 16.86 |
| 7.62 | 0.07 | 11.6 | 645.19 | 1.17 |
| 8.47 | 0.07 | 10.44 | 6897.59 | 12.5 |
| 8.96 | 0.07 | 9.87 | 1753.16 | 3.18 |
| 9.89 | 0.07 | 8.95 | 55179.41 | 100 |
| 10.55 | 0.07 | 8.38 | 4926.85 | 8.93 |
| 11.26 | 0.07 | 7.86 | 1383.55 | 2.51 |
| 12.58 | 0.07 | 7.04 | 14378.02 | 26.06 |
| 12.81 | 0.07 | 6.91 | 1158.16 | 2.1 |
| 12.94 | 0.07 | 6.84 | 1279.48 | 2.32 |
| 14.13 | 0.07 | 6.27 | 15192.69 | 27.53 |
| 14.28 | 0.05 | 6.2 | 6487.57 | 11.76 |
| 14.41 | 0.05 | 6.15 | 5949.89 | 10.78 |
| 14.73 | 0.07 | 6.01 | 17782.75 | 32.23 |
| 15.18 | 0.07 | 5.84 | 7354.25 | 13.33 |
| 15.3 | 0.05 | 5.79 | 3128.89 | 5.67 |
| 15.68 | 0.07 | 5.65 | 212.72 | 0.39 |
| 16.12 | 0.07 | 5.5 | 3183.26 | 5.77 |
| 16.55 | 0.07 | 5.36 | 6197.02 | 11.23 |
| 16.69 | 0.05 | 5.31 | 3313.49 | 6 |
| 16.91 | 0.1 | 5.24 | 27637.98 | 50.09 |
| 17.84 | 0.07 | 4.97 | 13395.18 | 24.28 |
| 17.99 | 0.05 | 4.93 | 8421.8 | 15.26 |
| 18.14 | 0.08 | 4.89 | 15082.36 | 27.33 |
| 18.6 | 0.1 | 4.77 | 815.82 | 1.48 |
| 19.26 | 0.1 | 4.61 | 9833.21 | 17.82 |
| 19.5 | 0.05 | 4.55 | 1756.59 | 3.18 |
| 19.67 | 0.07 | 4.51 | 4545.15 | 8.24 |
| 19.99 | 0.1 | 4.44 | 49351.1 | 89.44 |
| 20.28 | 0.07 | 4.38 | 8026.6 | 14.55 |
| 20.43 | 0.07 | 4.35 | 7142.96 | 12.94 |
| 20.76 | 0.08 | 4.28 | 7327.01 | 13.28 |
| 20.95 | 0.08 | 4.24 | 4807.28 | 8.71 |
| 22.07 | 0.1 | 4.03 | 8737.97 | 15.84 |
| 22.53 | 0.08 | 3.95 | 4115.78 | 7.46 |
| 22.91 | 0.06 | 3.88 | 5134.71 | 9.31 |
| 22.99 | 0.1 | 3.87 | 7045.44 | 12.77 |
| 23.07 | 0.06 | 3.86 | 5385.66 | 9.76 |
| 23.55 | 0.16 | 3.78 | 2669.22 | 4.84 |
| 23.87 | 0.12 | 3.72 | 2835.96 | 5.14 |
| 24.12 | 0.1 | 3.69 | 1074.27 | 1.95 |
| 24.41 | 0.06 | 3.64 | 2057.53 | 3.73 |
| 24.56 | 0.08 | 3.62 | 3426.35 | 6.21 |
| 25.31 | 0.1 | 3.52 | 3147.99 | 5.71 |
| 25.52 | 0.16 | 3.49 | 4084.76 | 7.4 |
| 26.01 | 0.06 | 3.42 | 2372.54 | 4.3 |
| 26.14 | 0.08 | 3.41 | 3614.13 | 6.55 |
| 26.4 | 0.1 | 3.37 | 4736.43 | 8.58 |
| 26.48 | 0.06 | 3.37 | 3940.25 | 7.14 |
| 26.67 | 0.12 | 3.34 | 3764.94 | 6.82 |
| 26.84 | 0.08 | 3.32 | 2746.76 | 4.98 |
| 27.14 | 0.1 | 3.28 | 969.83 | 1.76 |
| 27.78 | 0.14 | 3.21 | 2670.53 | 4.84 |
| 28.46 | 0.1 | 3.13 | 2097.97 | 3.8 |
| 28.65 | 0.1 | 3.11 | 2238.56 | 4.06 |
| 29.23 | 0.08 | 3.05 | 459.52 | 0.83 |
| 29.46 | 0.1 | 3.03 | 1595.7 | 2.89 |
| 29.69 | 0.14 | 3.01 | 3821.37 | 6.93 |
| 29.96 | 0.08 | 2.98 | 759.4 | 1.38 |
| 30.15 | 0.06 | 2.96 | 569.8 | 1.03 |
| 30.47 | 0.08 | 2.93 | 1977.99 | 3.58 |
| 30.73 | 0.08 | 2.91 | 986.41 | 1.79 |
| 31.33 | 0.14 | 2.85 | 1079.1 | 1.96 |
| 31.62 | 0.06 | 2.83 | 517.61 | 0.94 |
| 31.72 | 0.08 | 2.82 | 438.04 | 0.79 |
| 32.01 | 0.04 | 2.79 | 420.43 | 0.76 |
| 32.18 | 0.1 | 2.78 | 953.95 | 1.73 |
| 32.55 | 0.08 | 2.75 | 677.51 | 1.23 |
| 32.77 | 0.08 | 2.73 | 276.45 | 0.5 |
| 33.03 | 0.08 | 2.71 | 315.82 | 0.57 |
| 33.44 | 0.06 | 2.68 | 411.23 | 0.75 |
| 33.83 | 0.08 | 2.65 | 1499.7 | 2.72 |
| 33.94 | 0.06 | 2.65 | 973.31 | 1.76 |
| 34.22 | 0.08 | 2.62 | 546.22 | 0.99 |
| 34.46 | 0.1 | 2.6 | 882.45 | 1.6 |
| 34.83 | 0.08 | 2.57 | 1052.22 | 1.91 |
| 35.23 | 0.08 | 2.55 | 187.37 | 0.34 |
| 35.44 | 0.08 | 2.53 | 379.93 | 0.69 |
| 35.69 | 0.12 | 2.51 | 641.66 | 1.16 |
| 36.32 | 0.12 | 2.47 | 402.71 | 0.73 |
| 36.45 | 0.06 | 2.47 | 464.29 | 0.84 |
| 36.86 | 0.08 | 2.44 | 210.46 | 0.38 |
| 37.1 | 0.06 | 2.42 | 356.4 | 0.65 |
| 37.43 | 0.08 | 2.4 | 640.03 | 1.16 |
| 37.53 | 0.08 | 2.39 | 666.29 | 1.21 |
| 37.78 | 0.08 | 2.38 | 371.18 | 0.67 |
| 38.06 | 0.1 | 2.36 | 221.59 | 0.4 |
| 38.82 | 0.06 | 2.32 | 676.71 | 1.23 |
| 39.08 | 0.08 | 2.3 | 538.12 | 0.98 |
| 39.38 | 0.08 | 2.29 | 1279.85 | 2.32 |
| 39.63 | 0.04 | 2.27 | 864.52 | 1.57 |

### ¹H NMR Data

¹H NMR—0.02 mol EtOH (partially desolvated due to drying)

¹H NMR (400 MHz, DMSO-d6) δ ppm 0.73-0.98 (m, 2H) 0.98-1.12 (m, 1H) 1.00-1.10 (m, 1H) 1.00-1.10 (m, 1H) 1.12-1.20 (m, 1H) 1.20-1.29 (m, 1H) 1.29-1.45 (m, 1H) 1.45-1.56 (m, 1H) 1.56-1.70 (m, 2H) 1.70-1.82 (m, 1H) 1.85 (br s, 1H) 1.91 (s, 1H) 2.00 (br d, J=13.27 Hz, 1H) 2.12-2.29 (m, 2H) 2.33 (dt, J=3.65, 1.80 Hz, 1H) 2.52-2.62 (m, 1H) 2.63-2.85 (m, 2H) 2.86-3.00 (m, 1H) 3.00-3.14 (m, 1H) 3.14-3.28 (m, 2H) 3.35-3.59 (m, 1H) 3.50-3.62 (m, 1H) 3.78-3.90 (m, 1H) 3.91-4.16 (m, 3H) 4.17-4.40 (m, 1H) 4.50-4.84 (m, 2H) 5.32-5.52 (m, 2H) 5.59 (br s, 2H) 6.81 (br s, 1H) 6.90-7.09 (m, 1H) 7.09-7.21 (m, 2H) 7.27 (br dd, J=8.58, 2.24 Hz, 2H) 7.67 (d, J=8.58 Hz, 1H).

TABLE 19
X-ray Single Structure Data

| Crystal system | Primitive Monoclinic | |
|---|---|---|
| Extinction Symbol | P 1 2₁ 1 | |
| Space group | P2₁(4) | |
| Unit cell dimensions | a = 12.062 Å | α = 90° |
| | b = 14.057 Å | β = 106.24° |
| | c = 14.379 Å | γ = 90° |

| | |
|---|---|
| Volume | 2314.6 Å³ |
| Chiral Contents? | Chiral |

TABLE 20
X-ray Single Crystal Structure Data

| Wavelength | 0.710730 Å | |
|---|---|---|
| Crystal system | Monoclinic | |
| Extinction Symbol | P 2₁ | |
| Space group | P2₁(4) | |
| Unit cell dimensions | a = 12.0521 (16) Å | α = 90° |
| | b = 13.8878 (18) Å | β = 105.576 (2)° |
| | c = 14.1166 (18) Å | γ = 90° |

| | |
|---|---|
| Volume | 2277.1 (5) Å³ |
| Z | 2 |
| Density (calculated) | 1.252 g/cm³ |
| Absolute structure parameter | −0.08(5) |

### Example 10: AMG 397 Hydrate Form 9

AMG 397 hydrate Form 9 was prepared by charging AMG 397 with anhydrous MeOH or MeOH/H₂O (94:6) and stirring at room temperature for 2 weeks. Isolated solids were Form 9 Hydrate.

TABLE 21
XRPD Data Table

| Pos. [°2θ] | FWHM [°2θ] | d-spacing [Å] | Height [cts] | Rel. Int. [%] |
|---|---|---|---|---|
| 2.20 | 0.67 | 40.12 | 99.97 | 0.15 |
| 3.28 | 0.2 | 26.9 | 127.55 | 0.19 |
| 6.4 | 0.07 | 13.81 | 17604.21 | 25.69 |
| 7.76 | 0.07 | 11.4 | 2250.69 | 3.29 |
| 8.79 | 0.07 | 10.06 | 11582.64 | 16.91 |
| 9.03 | 0.07 | 9.79 | 2315.45 | 3.38 |
| 10.04 | 0.08 | 8.81 | 68513.92 | 100 |
| 10.86 | 0.07 | 8.14 | 11329.68 | 16.54 |
| 11.2 | 0.08 | 7.9 | 2850.16 | 4.16 |
| 12.74 | 0.08 | 6.95 | 13207.54 | 19.28 |
| 13.3 | 0.07 | 6.66 | 809.54 | 1.18 |
| 14.29 | 0.13 | 6.2 | 17064.76 | 24.91 |
| 14.76 | 0.07 | 6 | 11241.3 | 16.41 |
| 14.94 | 0.07 | 5.93 | 17293.22 | 25.24 |
| 15.33 | 0.05 | 5.78 | 2396.43 | 3.5 |
| 15.51 | 0.1 | 5.71 | 7148.45 | 10.43 |
| 16.61 | 0.12 | 5.34 | 4973.68 | 7.26 |
| 16.82 | 0.05 | 5.27 | 9414.36 | 13.74 |
| 17 | 0.08 | 5.22 | 27929.15 | 40.76 |
| 17.75 | 0.1 | 5 | 20426.57 | 29.81 |
| 18.12 | 0.07 | 4.9 | 8207.72 | 11.98 |
| 18.24 | 0.07 | 4.86 | 5107.73 | 7.46 |
| 18.46 | 0.07 | 4.81 | 1678.21 | 2.45 |
| 18.78 | 0.08 | 4.73 | 13101.17 | 19.12 |
| 19.32 | 0.1 | 4.59 | 13704.12 | 20 |
| 20.03 | 0.03 | 4.43 | 5369.52 | 7.84 |
| 20.23 | 0.1 | 4.39 | 46419.55 | 67.75 |
| 20.71 | 0.08 | 4.29 | 4422.45 | 6.45 |
| 20.94 | 0.07 | 4.24 | 4456.64 | 6.5 |
| 21.13 | 0.08 | 4.21 | 5092.46 | 7.43 |
| 21.85 | 0.08 | 4.07 | 609.89 | 0.89 |
| 22.26 | 0.12 | 3.99 | 8384.92 | 12.24 |
| 22.53 | 0.07 | 3.95 | 1526.71 | 2.23 |
| 22.74 | 0.07 | 3.91 | 3506.15 | 5.12 |
| 22.94 | 0.08 | 3.88 | 5923.6 | 8.65 |
| 23.14 | 0.12 | 3.84 | 5354.36 | 7.81 |
| 23.42 | 0.1 | 3.8 | 7387.31 | 10.78 |
| 23.64 | 0.07 | 3.76 | 1840.23 | 2.69 |
| 24.3 | 0.13 | 3.66 | 795.9 | 1.16 |
| 24.74 | 0.12 | 3.6 | 4412.54 | 6.44 |
| 25.26 | 0.12 | 3.53 | 2935.8 | 4.28 |
| 25.64 | 0.1 | 3.47 | 2394.94 | 3.5 |
| 25.96 | 0.1 | 3.43 | 4469.34 | 6.52 |
| 26.16 | 0.08 | 3.41 | 3125.3 | 4.56 |
| 26.45 | 0.03 | 3.37 | 2517.93 | 3.68 |
| 26.59 | 0.12 | 3.35 | 6149.45 | 8.98 |
| 26.86 | 0.08 | 3.32 | 2055.77 | 3 |
| 27.13 | 0.12 | 3.29 | 4619.56 | 6.74 |
| 27.35 | 0.07 | 3.26 | 1880.89 | 2.75 |
| 27.58 | 0.1 | 3.23 | 2578.26 | 3.76 |
| 27.69 | 0.08 | 3.23 | 1984.23 | 2.9 |
| 28.06 | 0.12 | 3.18 | 2622.05 | 3.83 |
| 28.45 | 0.08 | 3.13 | 1618.29 | 2.36 |
| 28.52 | 0.04 | 3.13 | 1386.26 | 2.02 |
| 28.77 | 0.12 | 3.1 | 2602.09 | 3.8 |
| 29.51 | 0.08 | 3.02 | 1882.97 | 2.75 |
| 29.74 | 0.12 | 3 | 3487.61 | 5.09 |
| 30.01 | 0.06 | 2.97 | 2152.03 | 3.14 |
| 30.12 | 0.08 | 2.96 | 1843.88 | 2.69 |
| 30.44 | 0.06 | 2.93 | 1138.48 | 1.66 |
| 30.68 | 0.14 | 2.91 | 3386.05 | 4.94 |
| 31.25 | 0.16 | 2.86 | 1242.54 | 1.81 |
| 31.62 | 0.16 | 2.83 | 1585.75 | 2.31 |
| 32.12 | 0.1 | 2.78 | 539.02 | 0.79 |
| 32.37 | 0.16 | 2.76 | 1257.02 | 1.83 |
| 33.16 | 0.18 | 2.7 | 1347.05 | 1.97 |
| 33.6 | 0.1 | 2.66 | 1021.94 | 1.49 |
| 33.91 | 0.06 | 2.64 | 1073.46 | 1.57 |
| 34.03 | 0.08 | 2.63 | 1116.53 | 1.63 |
| 34.2 | 0.06 | 2.63 | 808.33 | 1.18 |
| 34.54 | 0.08 | 2.59 | 870.41 | 1.27 |
| 34.65 | 0.06 | 2.59 | 965.08 | 1.41 |
| 34.81 | 0.08 | 2.57 | 1139.95 | 1.66 |
| 35.02 | 0.1 | 2.56 | 1227.14 | 1.79 |
| 35.38 | 0.06 | 2.53 | 478.71 | 0.7 |
| 36.27 | 0.2 | 2.47 | 1079.48 | 1.58 |
| 36.68 | 0.08 | 2.45 | 521.99 | 0.76 |
| 37.05 | 0.12 | 2.42 | 701.46 | 1.02 |
| 37.29 | 0.08 | 2.41 | 559.05 | 0.82 |
| 37.64 | 0.1 | 2.39 | 815.36 | 1.19 |
| 37.97 | 0.1 | 2.37 | 561.89 | 0.82 |
| 38.46 | 0.1 | 2.34 | 484.1 | 0.71 |
| 38.93 | 0.1 | 2.31 | 470.24 | 0.69 |

### 1H NMR Data

1H NMR (400 MHz, DMSO-d6) δ ppm 0.84-1.06 (m, 2H) 1.06-1.26 (m, 3H) 1.26-1.49 (m, 3H) 1.49-1.88 (m, 6H) 1.91 (s, 1H) 1.95-2.08 (m, 2H) 2.08-2.34 (m, 4H) 2.39-2.48 (m, 1H) 2.54-2.82 (m, 4H) 2.86-3.01 (m, 2H) 3.01-3.16 (m, 1H) 3.16-3.26 (m, 3H) 3.55 (br d, J=14.07 Hz, 2H) 3.65-3.78 (m, 1H) 3.86 (br d, J=14.39 Hz, 2H) 3.91-4.04 (m, 2H) 4.04-4.21 (m, 1H) 5.32-5.52 (m, 2H) 5.61 (br d, J=16.04 Hz, 2H) 6.72-6.91 (m, 2H) 6.91-7.12 (m, 2H) 7.12-7.21 (m, 2H) 7.27 (dd, J=8.50, 2.26 Hz, 1H) 7.67 (d, J=8.58 Hz, 1H).

TABLE 22
X-ray Single Structure Data

| Crystal system | Primitive Monoclinic | |
|---|---|---|
| Extinction Symbol | P 1 2₁ 1 | |
| Space group | P2₁(4) | |
| Unit cell dimensions | a = 11.727 Å | α = 90° |
| | b = 13.872 Å | β = 103.94° |
| | c = 14.210 Å | γ = 90° |

| | |
|---|---|
| Volume | 2243.7 Å³ |
| Chiral Contents? | Chiral |

### Example 11: AMG 397 Hydrate Form 10

AMG 397 hydrate Form 10 was prepared by extracting AMG 397 from 100 mg drug product tablet using Me-THF and solvent exchange to ethanol, then adding NaOH to form the sodium salt. The slurry was filtered to collect the hazy filtrate, and some crystalline material was noted growing on the sides of the flask holding the EtOH filtrate. ICP-MS analysis confirmed that the compound was in free base form.

TABLE 23
XRPD Data Table

| Pos. [°2θ] | FWHM [°2θ] | d-spacing [Å] | Height [cts] | Rel. Int. [%] |
|---|---|---|---|---|
| 6.4105 | 0.1428 | 13.77674 | 1464.48 | 26.31 |
| 6.6433 | 0.1224 | 13.29456 | 1076.79 | 19.34 |
| 7.4385 | 0.0612 | 11.87501 | 43.36 | 0.78 |
| 8.5442 | 0.0612 | 10.34053 | 641.98 | 11.53 |
| 8.7417 | 0.0612 | 10.10731 | 408.41 | 7.34 |
| 9.1269 | 0.1632 | 9.68157 | 153.14 | 2.75 |
| 10.0536 | 0.1632 | 8.7912 | 4340.99 | 77.98 |
| 10.7198 | 0.1428 | 8.24627 | 631.56 | 11.35 |
| 11.4839 | 0.1632 | 7.6993 | 76.1 | 1.37 |
| 12.0899 | 0.2448 | 7.31466 | 30.15 | 0.54 |
| 12.812 | 0.1632 | 6.904 | 1223.15 | 21.97 |
| 13.9066 | 0.0408 | 6.36292 | 88.09 | 1.58 |
| 14.3991 | 0.1632 | 6.14637 | 2396.4 | 43.05 |
| 14.9389 | 0.102 | 5.92547 | 1800.51 | 32.34 |
| 15.3622 | 0.1836 | 5.76315 | 1104.58 | 19.84 |
| 16.2813 | 0.1632 | 5.43982 | 638.41 | 11.47 |
| 16.6895 | 0.1224 | 5.3077 | 1330.22 | 23.9 |
| 17.0864 | 0.1836 | 5.18528 | 3607.99 | 64.81 |
| 17.9166 | 0.0816 | 4.94683 | 1779.74 | 31.97 |
| 18.2564 | 0.1224 | 4.85552 | 2708.7 | 48.66 |
| 19.3659 | 0.1428 | 4.57977 | 884.89 | 15.9 |
| 19.7818 | 0.1224 | 4.48442 | 719.9 | 12.93 |
| 20.2119 | 0.1224 | 4.38995 | 5202.76 | 93.46 |
| 20.306 | 0.1224 | 4.36981 | 5566.8 | 100 |
| 21.0616 | 0.1632 | 4.21472 | 1137.15 | 20.43 |
| 22.3071 | 0.102 | 3.98213 | 1049.35 | 18.85 |
| 22.7144 | 0.1632 | 3.91163 | 556.79 | 10 |
| 23.1774 | 0.204 | 3.83454 | 1215.45 | 21.83 |
| 23.6824 | 0.2448 | 3.75389 | 234.42 | 4.21 |
| 23.8197 | 0.0612 | 3.73257 | 146.49 | 2.63 |
| 23.9841 | 0.0612 | 3.70735 | 94.15 | 1.69 |
| 24.1647 | 0.0612 | 3.68006 | 94.73 | 1.7 |
| 24.7162 | 0.0816 | 3.59917 | 526.56 | 9.46 |
| 25.2123 | 0.1632 | 3.52946 | 124.04 | 2.23 |
| 25.7138 | 0.204 | 3.46176 | 698.69 | 12.55 |
| 26.1588 | 0.1632 | 3.40387 | 290.88 | 5.23 |
| 26.5316 | 0.204 | 3.35687 | 592.74 | 10.65 |
| 26.9101 | 0.2856 | 3.31051 | 824.6 | 14.81 |
| 27.3284 | 0.0612 | 3.26079 | 212.76 | 3.82 |
| 28.0846 | 0.2448 | 3.17468 | 401.17 | 7.21 |
| 28.6763 | 0.3672 | 3.11051 | 332.49 | 5.97 |
| 29.6444 | 0.1224 | 3.0111 | 168.16 | 3.02 |
| 29.8368 | 0.1428 | 2.99212 | 508.16 | 9.13 |
| 30.7204 | 0.3264 | 2.90804 | 188.14 | 3.38 |
| 31.6051 | 0.3264 | 2.82862 | 159.17 | 2.86 |
| 31.8478 | 0.0612 | 2.80762 | 83.1 | 1.49 |
| 32.2408 | 0.1632 | 2.77429 | 82.43 | 1.48 |

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure herein.

All patents, publications and references cited herein are hereby fully incorporated by reference. In case of conflict between the present disclosure and incorporated patents, publications and references, the present disclosure should control.

### EMBODIMENTS

The foregoing disclosure may be better understood through the following embodiments.

Embodiment 1. A crystalline form of AMG 397 as a hydrate, characterized by solid state ¹³C NMR peaks at 5.65, 15.29, 18.06, 21.54, 24.20, 24.87, 28.91, 29.87, 36.86, 37.74, 39.09, 43.79, 44.59, 48.25, 49.01, 51.76, 54.33, 55.45, 57.50, 60.39, 64.99, 66.40, 80.11, 82.55, 83.01, 115.39, 121.81, 124.57, 127.61, 129.92, 132.04, 133.60, 135.32, 140.41, 142.61, 143.54, 153.09, 173.18, and 174.17±0.5 ppm (“Form 2 hydrate”).

Embodiment 2. The crystalline form of embodiment 1, further characterized by XRPD pattern peaks at 6.2, 7.4, and 15.7±0.2° 2θ using Cu Kα radiation.

Embodiment 3. The crystalline form of embodiment 2, further characterized by XRPD pattern peaks at 11.4, 16.0, 18.0, and 22.1±0.2° 2θ using Cu Kα radiation.

Embodiment 4. The crystalline form of embodiment 3, further characterized by XRPD pattern peaks at 10.2, 10.6, 11.9, 17.1, 18.5, 19.2, 19.7, 20.3, 20.9, and 21.8±0.2° 2θ using Cu Kα radiation.

Embodiment 5. The crystalline form of any one of embodiments 1 to 4, having an XRPD pattern substantially as shown in FIG. 11.

Embodiment 6. The crystalline form of any one of embodiments 1 to 5, having an endothermic transition at 245° C. to 251° C., as measured by differential scanning calorimetry.

Embodiment 7. The crystalline form of embodiment 6, wherein the endothermic transition is at 248° C.±3° C.

Embodiment 8. The crystalline form of any one of embodiments 1 to 7 having a thermogravimetric analysis (“TGA”) substantially as shown in FIG. 13.

Embodiment 9. A crystalline form of AMG 397 as a hydrate, characterized by solid state ¹³C NMR peaks at 7.07, 17.2, 21.14, 22.75, 23.74, 27.01, 27.79, 29.13, 30.12, 32.09, 33.0, 35.45, 37.96, 45.21, 45.88, 50.0, 54.43, 55.23, 57.5, 59.23, 61.66, 63.31, 64.14, 69.06, 76.48, 82.72, 116.84, 119.24, 121.1, 126.62, 130.68, 132.8, 136.76, 139.39, 140.98, 141.7, 151.61, 172.8, and 173.61±0.5 ppm (“Form 3 hydrate”).

Embodiment 10. The crystalline form of embodiment 9, further characterized by XRPD pattern peaks at 13.6, 15.4, and 18.1±0.2° 2θ using Cu Kα radiation.

Embodiment 11. The crystalline form of embodiment 10, further characterized by XRPD pattern peaks at 16.5, 18.9, 21.9, 22.6, and 24.2±0.2° 2θ using Cu Kα radiation.

Embodiment 12. The crystalline form of embodiment 11, further characterized by XRPD pattern peaks at 12.3, 13.0, 16.0, 16.8, 17.5, 18.5, 19.5, 23.0, 27.2, and 28.0±0.2° 2θ using Cu Kα radiation.

Embodiment 13. The crystalline form of any one of embodiments 9 to 12, having an XRPD pattern substantially as shown in FIG. 16.

Embodiment 14. The crystalline form of any one of embodiments 9 to 13, having an endothermic transition at 234° C. to 240° C., as measured by differential scanning calorimetry.

Embodiment 15. The crystalline form of embodiment 14, wherein the endothermic transition is at 237° C.±3° C.

Embodiment 16. The crystalline form of any one of embodiments 9 to 15, having a thermogravimetric analysis (“TGA”) substantially as shown in FIG. 18.

Embodiment 17. A crystalline form of AMG 397 anhydrous, characterized by solid state ¹³C NMR peaks at 5.55, 17.86, 24.02, 24.95, 29.56, 37.70, 44.44, 47.61, 48.86, 51.26, 54.92, 56.72, 57.48, 58.58, 64.86, 82.34, 114.99, 121.30, 127.31, 131.61, 133.04, 135.02, 139.77, 141.92, 152.71, and 173.08±0.5 ppm (“Form 4 anhydrous”).

Embodiment 18. The crystalline form of embodiment 17, further characterized by XRPD pattern peaks at 11.2, 15.8, and 19.3±0.2° 2θ using Cu Kα radiation.

Embodiment 19. The crystalline form of embodiment 18, further characterized by XRPD pattern peaks at 12.9, 14.4, 16.8, and 18.2±0.2° 2θ using Cu Kα radiation.

Embodiment 20. The crystalline form of embodiment 19, further characterized by XRPD pattern peaks at 10.7, 13.4, 15.4, 17.3, 18.5, 20.1, 20.4, 20.6, 21.7, 22.3, 24.9, and 26.5±0.2° 2θ using Cu Kα radiation.

Embodiment 21. The crystalline form of any one of embodiments 17 to 20, having an XRPD pattern substantially as shown in FIG. 21.

Embodiment 22. The crystalline form of any one of embodiments 17 to 21, having an endothermic transition at 239° C. to 245° C., as measured by differential scanning calorimetry.

Embodiment 23. The crystalline form of embodiment 22, wherein the endothermic transition is at 242° C.±3° C.

Embodiment 24. The crystalline form of any one of embodiments 17 to 23, having a thermogravimetric analysis (“TGA”) substantially as shown in FIG. 23.

Embodiment 25. A crystalline form of AMG 397 as a hydrate, characterized by solid state ¹³C NMR peaks at 5.90, 15.93, 21.71, 24.33, 24.99, 25.92, 28.37, 29.16, 30.25, 31.00, 37.10, 39.31, 44.09, 48.49, 49.30, 51.99, 54.58, 55.81, 56.34, 57.73, 60.59, 66.60, 80.42, 83.22, 115.55, 122.14, 124.75, 127.82, 130.10, 132.40, 133.76, 140.62, 142.89, 143.63, 153.36, and 174.41±0.5 ppm (“Form 5 hydrate”).

Embodiment 26. The crystalline form of embodiment 25, further characterized by XRPD pattern peaks at 15.8, 16.8, and 19.4±0.2° 2θ using Cu Kα radiation.

Embodiment 27. The crystalline form of embodiment 26, further characterized by XRPD pattern peaks at 11.3, 14.5, 18.2, 20.6, and 22.3±0.2° 2θ using Cu Kα radiation.

Embodiment 28. The crystalline form of embodiment 27, further characterized by XRPD pattern peaks at 6.4, 10.7, 12.5, 13.0, 13.5, 16.1, 17.3, 18.6, 19.8, 20.1, 21.8, 24.9, and 26.6±0.2° 2θ using Cu Kα radiation.

Embodiment 29. The crystalline form of any one of embodiments 25 to 28, having an XRPD pattern substantially as shown in FIG. 26.

Embodiment 30. The crystalline form of any one of embodiments 25 to 29, having an endothermic transition at 234° C. to 240° C., as measured by differential scanning calorimetry.

Embodiment 31. The crystalline form of embodiment 30, wherein the endothermic transition is at 237° C.±3° C.

Embodiment 32. The crystalline form of any one of embodiments 25 to 31, having a thermogravimetric analysis (“TGA”) substantially as shown in FIG. 28.

Embodiment 33. A crystalline form of AMG 397 anhydrous, characterized by XRPD pattern peaks at 8.3, 15.7, 16.0, 18.6, and 20.1±0.2° 2θ using Cu Kα radiation (“Form 6 anhydrous”).

Embodiment 34. The crystalline form of embodiment 33, further characterized by XRPD pattern peaks at 11.0, 12.5, 14.0, 18.4, 19.5, and 23.9±0.2° 2θ using Cu Kα radiation.

Embodiment 35. The crystalline form of embodiment 34, further characterized by XRPD pattern peaks at 8.6, 13.1, 14.3, 14.7, 15.4, 17.2, 17.6, 18.1, 21.9, 22.2, 22.5, 22.7, and 28.2±0.2° 2θ using Cu Kα radiation.

Embodiment 36. The crystalline form of any one of embodiments 33 to 35, having an XRPD pattern substantially as shown in FIG. 30.

Embodiment 37. The crystalline form of any one of embodiments 33 to 36, having an endothermic transition at 231° C. to 237° C., as measured by differential scanning calorimetry.

Embodiment 38. The crystalline form of embodiment 46, wherein the endothermic transition is at 234° C.±3° C.

Embodiment 39. The crystalline form of any one of embodiments 33 to 38, having a thermogravimetric analysis (“TGA”) substantially as shown in FIG. 32.

Embodiment 40. A crystalline form of AMG 397 as a hydrate, characterized by XRPD pattern peaks at 8.3, 10.7, and 10.8±0.2° 2θ using Cu Kα radiation (“Form 7 hydrate”).

Embodiment 41. The crystalline form of embodiment 40, further characterized by XRPD pattern peaks at 1.0, 12.5, 13.9, 16.8, 17.3, 18.7, and 19.3±0.2° 2θ using Cu Kα radiation.

Embodiment 42. The crystalline form of embodiment 41, further characterized by XRPD pattern peaks at 6.3, 13.7, 14.2, 16.6, 18.9, 20.5, 20.6, 21.1, 21.7, 23.6, and 23.8±0.2° 2θ using Cu Kα radiation.

Embodiment 43. The crystalline form of any one of embodiments 40 to 42, having an XRPD pattern substantially as shown in FIG. 34.

Embodiment 44. The crystalline form of any one of embodiments 40 to 43, having an endothermic transition at 216° C. to 224° C., as measured by differential scanning calorimetry.

Embodiment 45. The crystalline form of embodiment 44, wherein the endothermic transition is at 220° C.±3° C.

Embodiment 46. The crystalline form of any one of embodiments 40 to 45, having a thermogravimetric analysis (“TGA”) substantially as shown in FIG. 36.

Embodiment 47. A crystalline form of AMG 397 as an ethanol solvate, characterized by XRPD pattern peaks at 9.9, 16.9, and 20.0±0.2° 2θ using Cu Kα radiation (“Form 8 ethanol solvate”).

Embodiment 48. The crystalline form of embodiment 47, further characterized by XRPD pattern peaks at 12.6, 14.1, 14.7, 17.8, and 18.1±0.2° 2θ using Cu Kα radiation.

Embodiment 49. The crystalline form of embodiment 48, further characterized by XRPD pattern peaks at 6.4, 8.5, 14.3, 14.4, 15.2, 16.6, 19.3, 20.3, 20.4, 20.8, 22.1, and 23.0±0.2° 2θ using Cu Kα radiation.

Embodiment 50. The crystalline form of any one of embodiments 47 to 49, having an XRPD pattern substantially as shown in FIG. 38.

Embodiment 51. The crystalline form of any one of embodiments 47 to 50, having an endothermic transition at 64° C. to 70° C. and 233° C. to 239° C., as measured by differential scanning calorimetry.

Embodiment 52. The crystalline form of embodiment 51, wherein the endothermic transition is at 67° C. and 236° C.±3° C.

Embodiment 53. The crystalline form of any one of embodiments 47 to 52, having a thermogravimetric analysis (“TGA”) substantially as shown in FIG. 40.

Embodiment 54. The crystalline form of any one of embodiments 47 to 53, having a single crystal structure substantially as shown in FIG. 41.

Embodiment 55. A crystalline form of AMG 397 as a hydrate, characterized by XRPD pattern peaks at 10.0, 17.0, and 20.2±0.2° 2θ using Cu Kα radiation (“Form 9 hydrate”).

Embodiment 56. The crystalline form of embodiment 55, further characterized by XRPD pattern peaks at 6.4, 14.3, 14.9, 17.8, and 19.3±0.2° 2θ using Cu Kα radiation.

Embodiment 57. The crystalline form of embodiment 56, further characterized by XRPD pattern peaks at 8.8, 10.9, 12.7, 14.8, 15.5, 16.8, 18.1, 18.8, 22.3, and 23.4±0.2° 2θ using Cu Kα radiation.

Embodiment 58. The crystalline form of any one of embodiments 55 to 57, having an XRPD pattern substantially as shown in FIG. 42.

Embodiment 59. The crystalline form of any one of embodiments 55 to 58, having an endothermic transition at 231° C. to 237° C., as measured by differential scanning calorimetry.

Embodiment 60. The crystalline form of embodiment 59, wherein the endothermic transition is at 234° C.±3° C.

Embodiment 61. The crystalline form of any one of embodiments 55 to 60, having a thermogravimetric analysis (“TGA”) substantially as shown in FIG. 44.

Embodiment 62. A crystalline form of AMG 397 as a hydrate, characterized by XRPD pattern peaks at 10.1, 20.2, 20.3±0.2° 2θ using Cu Kα radiation (“Form 10 hydrate”).

Embodiment 63. The crystalline form of embodiment 62, further characterized by XRPD pattern peaks at 14.4, 14.9, 17.1, 17.9, and 18.3±0.2° 2θ using Cu Kα radiation.

Embodiment 64. The crystalline form of embodiment 63, further characterized by XRPD pattern peaks at 6.4, 6.6, 8.5, 10.7, 12.8, 15.4, 16.3, 16.7, 19.4, 19.8, 21.1, 22.3, 23.2, 25.7, 26.5, and 26.9±0.2° 2θ using Cu Kα radiation.

Embodiment 65. The crystalline form of any one of embodiments 62 to 64, having an XRPD pattern substantially as shown in FIG. 45.

Embodiment 66. The crystalline form of any one of embodiments 62 to 65, having an endothermic transition at 230° C. to 236° C., as measured by differential scanning calorimetry.

Embodiment 67. The crystalline form of embodiment 66, wherein the endothermic transition is at 233° C.±3° C.

Embodiment 68. The crystalline form of any one of embodiments 62 to 67, having a thermogravimetric analysis (“TGA”) substantially as shown in FIG. 47.

Embodiment 69. A pharmaceutical formulation comprising the crystalline form of any one of embodiments 1 to 68 and a pharmaceutically acceptable excipient.

Embodiment 70. A method of treating a subject suffering from cancer, comprising administering to the subject a therapeutically effective amount of the crystalline form of any one of embodiments 1 to 68 or the pharmaceutical formulation of embodiment 69.

Embodiment 71. The method of embodiment 70, wherein the cancer is multiple myeloma, non-Hodgkin's lymphoma, or acute myeloid leukemia.

### OTHER EMBODIMENTS

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

1. A crystalline form of AMG 397:
(i) as a hydrate, characterized by solid state ¹³C NMR peaks at 5.65, 15.29, 18.06, 21.54, 24.20, 24.87, 28.91, 29.87, 36.86, 37.74, 39.09, 43.79, 44.59, 48.25, 49.01, 51.76, 54.33, 55.45, 57.50, 60.39, 64.99, 66.40, 80.11, 82.55, 83.01, 115.39, 121.81, 124.57, 127.61, 129.92, 132.04, 133.60, 135.32, 140.41, 142.61, 143.54, 153.09, 173.18, and 174.17±0.5 ppm (“Form 2 hydrate”);
(ii) as a hydrate, characterized by solid state ¹³C NMR peaks at 7.07, 17.2, 21.14, 22.75, 23.74, 27.01, 27.79, 29.13, 30.12, 32.09, 33.0, 35.45, 37.96, 45.21, 45.88, 50.0, 54.43, 55.23, 57.5, 59.23, 61.66, 63.31, 64.14, 69.06, 76.48, 82.72, 116.84, 119.24, 121.1, 126.62, 130.68, 132.8, 136.76, 139.39, 140.98, 141.7, 151.61, 172.8, and 173.61±0.5 ppm (“Form 3 hydrate”);
(iii) anhydrous, characterized by solid state ¹³C NMR peaks at 5.55, 17.86, 24.02, 24.95, 29.56, 37.70, 44.44, 47.61, 48.86, 51.26, 54.92, 56.72, 57.48, 58.58, 64.86, 82.34, 114.99, 121.30, 127.31, 131.61, 133.04, 135.02, 139.77, 141.92, 152.71, and 173.08±0.5 ppm (“Form 4 anhydrous”);
(iv) as a hydrate, characterized by solid state ¹³C NMR peaks at 5.90, 15.93, 21.71, 24.33, 24.99, 25.92, 28.37, 29.16, 30.25, 31.00, 37.10, 39.31, 44.09, 48.49, 49.30, 51.99, 54.58, 55.81, 56.34, 57.73, 60.59, 66.60, 80.42, 83.22, 115.55, 122.14, 124.75, 127.82, 130.10, 132.40, 133.76, 140.62, 142.89, 143.63, 153.36, and 174.41±0.5 ppm (“Form 5 hydrate”);
(v) anhydrous, characterized by XRPD pattern peaks at 8.3, 15.7, 16.0, 18.6, and 20.1±0.2° 2θ using Cu Kα radiation (“Form 6 anhydrous”);
(vi) as a hydrate, characterized by XRPD pattern peaks at 8.3, 10.7, and 10.8±0.2° 2θ using Cu Kα radiation (“Form 7 hydrate”);
(vii) as an ethanol solvate, characterized by XRPD pattern peaks at 9.9, 16.9, and 20.0±0.2° 2θ using Cu Kα radiation (“Form 8 ethanol solvate”);
(viii) as a hydrate, characterized by XRPD pattern peaks at 10.0, 17.0, and 20.2±0.2° 2θ using Cu Kα radiation (“Form 9 hydrate”); or
(ix) as a hydrate, characterized by XRPD pattern peaks at 10.1, 20.2, 20.3±0.2° 2θ using Cu Kα radiation (“Form 10 hydrate”).

2. The crystalline form of claim 1, wherein the AMG 397 Form 2 hydrate:
(i) is further characterized by XRPD pattern peaks at 6.2, 7.4, and 15.7±0.2° 2θ using Cu Kα radiation, optionally further characterized by XRPD pattern peaks at 11.4, 16.0, 18.0, and 22.1±0.2° 2θ using Cu Kα radiation, and optionally further characterized by XRPD pattern peaks at 10.2, 10.6, 11.9, 17.1, 18.5, 19.2, 19.7, 20.3, 20.9, and 21.8±0.2° 2θ using Cu Kα radiation;
(ii) has an XRPD pattern substantially as shown in FIG. 11;
(iii) has an endothermic transition at 245° C. to 251° C., as measured by differential scanning calorimetry; and/or
(iv) has a thermogravimetric analysis (“TGA”) substantially as shown in FIG. 13.

3. (canceled)

4. (canceled)

5. (canceled)

6. (canceled)

7. (canceled)

8. (canceled)

9. (canceled)

10. The crystalline form of claim 1, wherein the AMG 397 Form 3 hydrate:
(i) is further characterized by XRPD pattern peaks at 13.6, 15.4, and 18.1±0.2° 2θ using Cu Kα radiation, optionally further characterized by XRPD pattern peaks at 16.5, 18.9, 21.9, 22.6, and 24.2±0.2° 2θ using Cu Kα radiation, and optionally further characterized by XRPD pattern peaks at 12.3, 13.0, 16.0, 16.8, 17.5, 18.5, 19.5, 23.0, 27.2, and 28.0±0.2° 2θ using Cu Kα radiation;
(ii) has an XRPD pattern substantially as shown in FIG. 16;
(iii) has an endothermic transition at 234° C. to 240° C., as measured by differential scanning calorimetry; and/or
(iv) has a thermogravimetric analysis (“TGA”) substantially as shown in FIG. 18.

11. (canceled)

12. (canceled)

13. (canceled)

14. (canceled)

15. (canceled)

16. (canceled)

17. (canceled)

18. The crystalline form of claim 1, wherein the AMG 397 Form 4 anhydrous:
(i) is further characterized by XRPD pattern peaks at 11.2, 15.8, and 19.3±0.2° 2θ using Cu Kα radiation, optionally further characterized by XRPD pattern peaks at 12.9, 14.4, 16.8, and 18.2±0.2° 2θ using Cu Kα radiation, and optionally further characterized by XRPD pattern peaks at 10.7, 13.4, 15.4, 17.3, 18.5, 20.1, 20.4, 20.6, 21.7, 22.3, 24.9, and 26.5±0.2° 2θ using Cu Kα radiation;
(ii) has an XRPD pattern substantially as shown in FIG. 21;
(iii) has an endothermic transition at 239° C. to 245° C., as measured by differential scanning calorimetry; and/or
(iv) has a thermogravimetric analysis (“TGA”) substantially as shown in FIG. 23.

19. (canceled)

20. (canceled)

21. (canceled)

22. (canceled)

23. (canceled)

24. (canceled)

25. (canceled)

26. The crystalline form of claim 1, wherein the AMG 397 Form 5 hydrate:
(i) is further characterized by XRPD pattern peaks at 15.8, 16.8, and 19.4±0.2° 2θ using Cu Kα radiation, optionally further characterized by XRPD pattern peaks at 11.3, 14.5, 18.2, 20.6, and 22.3±0.2° 2θ using Cu Kα radiation, and optionally further characterized by Preliminary Amendment and Response to Notice of Insufficiency XRPD pattern peaks at 6.4, 10.7, 12.5, 13.0, 13.5, 16.1, 17.3, 18.6, 19.8, 20.1, 21.8, 24.9, and 26.6±0.2° 2θ using Cu Kα radiation;
(ii) has an XRPD pattern substantially as shown in FIG. 26;
(iii) has an endothermic transition at 234° C. to 240° C., as measured by differential scanning calorimetry; and/or
(iv) has a thermogravimetric analysis (“TGA”) substantially as shown in FIG. 28.

27. (canceled)

28. (canceled)

29. (canceled)

30. (canceled)

31. (canceled)

32. (canceled)

33. (canceled)

34. The crystalline form of claim 1, wherein the AMG 397 Form 6 anhydrous:
(i) is further characterized by XRPD pattern peaks at 11.0, 12.5, 14.0, 18.4, 19.5, and 23.9±0.2° 2θ using Cu Kα radiation, optionally further characterized by XRPD pattern peaks at 8.6, 13.1, 14.3, 14.7, 15.4, 17.2, 17.6, 18.1, 21.9, 22.2, 22.5, 22.7, and 28.2±0.2° 2θ using Cu Kα radiation;
(ii) has an XRPD pattern substantially as shown in FIG. 30;
(iii) has an endothermic transition at 231° C. to 237° C., as measured by differential scanning calorimetry; and/or
(iv) has a thermogravimetric analysis (“TGA”) substantially as shown in FIG. 32.

35. (canceled)

36. (canceled)

37. (canceled)

38. (canceled)

39. (canceled)

40. (canceled)

41. The crystalline form of claim 1, wherein the AMG 397 Form 7 hydrate:
(i) is further characterized by XRPD pattern peaks at 1.0, 12.5, 13.9, 16.8, 17.3, 18.7, and 19.3±0.2° 2θ using Cu Kα radiation, optionally further characterized by XRPD pattern peaks at 6.3, 13.7, 14.2, 16.6, 18.9, 20.5, 20.6, 21.1, 21.7, 23.6, and 23.8±0.2° 2θ using Cu Kα radiation;
(ii) has an XRPD pattern substantially as shown in FIG. 34;
(iii) has an endothermic transition at 216° C. to 224° C., as measured by differential scanning calorimetry; and/or
(iv) has a thermogravimetric analysis (“TGA”) substantially as shown in FIG. 36.

42. (canceled)

43. (canceled)

44. (canceled)

45. (canceled)

46. (canceled)

47. (canceled)

48. The crystalline form of claim 1, wherein the AMG 397 Form 8 ethanol solvate:
(i) is further characterized by XRPD pattern peaks at 12.6, 14.1, 14.7, 17.8, and 18.1±0.2° 2θ using Cu Kα radiation, optionally further characterized by XRPD pattern peaks at 6.4, 8.5, 14.3, 14.4, 15.2, 16.6, 19.3, 20.3, 20.4, 20.8, 22.1, and 23.0±0.2° 2θ using Cu Kα radiation;
(ii) has an XRPD pattern substantially as shown in FIG. 38;
(iii) has an endothermic transition at 64° C. to 70° C. and 233° C. to 239° C., as measured by differential scanning calorimetry; and/or
(iv) has a thermogravimetric analysis (“TGA”) substantially as shown in FIG. 40.

49. (canceled)

50. (canceled)

51. (canceled)

52. (canceled)

53. (canceled)

54. (canceled)

55. (canceled)

56. The crystalline form of claim 1, wherein the AMG 397 Form 9 hydrate:
(i) is further characterized by XRPD pattern peaks at 6.4, 14.3, 14.9, 17.8, and 19.3±0.2° 2θ using Cu Kα radiation, optionally further characterized by XRPD pattern peaks at 8.8, 10.9, 12.7, 14.8, 15.5, 16.8, 18.1, 18.8, 22.3, and 23.4±0.2° 2θ using Cu Kα radiation;
(ii) has an XRPD pattern substantially as shown in FIG. 42;
(iii) has an endothermic transition at 231° C. to 237° C., as measured by differential scanning calorimetry; and/or
(iv) has a thermogravimetric analysis (“TGA”) substantially as shown in FIG. 44.

57. (canceled)

58. (canceled)

59. (canceled)

60. (canceled)

61. (canceled)

62. (canceled)

63. The crystalline form of claim 1, wherein the AMG 397 Form 10 hydrate:
(i) is further characterized by XRPD pattern peaks at 14.4, 14.9, 17.1, 17.9, and 18.3±0.2° 2θ using Cu Kα radiation, optionally further characterized by XRPD pattern peaks at 6.4, 6.6, 8.5, 10.7, 12.8, 15.4, 16.3, 16.7, 19.4, 19.8, 21.1, 22.3, 23.2, 25.7, 26.5, and 26.9±0.2° 2θ using Cu Kα radiation;
(ii) has an XRPD Pattern substantially as shown in FIG. 45;
(iii) has an endothermic transition at 230° C. to 236° C., as measured by differential scanning calorimetry; and/or
(iv) has a thermogravimetric analysis (“TGA”) substantially as shown in FIG. 47.

64. (canceled)

65. (canceled)

66. (canceled)

67. (canceled)

68. (canceled)

69. A pharmaceutical formulation comprising the crystalline form of claim 1 and a pharmaceutically acceptable excipient.

70. A method of treating a subject suffering from cancer, comprising administering to the subject a therapeutically effective amount of the crystalline form of claim 1.

71. The method of claim 70, wherein the cancer is multiple myeloma, non-Hodgkin's lymphoma, or acute myeloid leukemia.
